(19) United States
(12) Patent Application Publication
(10) Pub. No.: US 2020/0085776 A1
(43) Pub. Date: Mar. 19, 2020

(54) Methods of Reducing C-Reactive Protein and/or Treating Cardiovascular Disease

(71) Applicant: CARDAX, INC., Honolulu, HI (US)

(72) Inventors: David G. Watumull, Honolulu, HI (US); David M. Watumull, Honolulu, HI (US); Jon L. Ruckle, Honolulu, HI (US); Timothy J. King, Honolulu, HI (US); Gilbert M. Rishton, Honolulu, HI (US); Andrew D. Hieber, Honolulu, HI (US); Henry L. Jackson, Honolulu, HI (US)

(21) Appl. No.: 16/566,800

(22) Filed: Sep. 10, 2019

Related U.S. Application Data
(60) Provisional application No. 62/729,152, filed on Sep. 10, 2018.

(51) Int. Cl. A61K 31/225; A61P 9/14; A61P 9/10

(52) U.S. Cl. CPC A61K 31/225; A61P 9/10; A61P 9/14

(57) ABSTRACT

Provided herein are methods of preventing or treating cardiovascular disease (CVD), methods of supporting cardiovascular health, or supporting inflammatory health in a subject by reducing C-reactive protein (CRP). In exemplary aspects, the method comprises administering astaxanthin to the subject at a therapeutically effective amount. In some aspects, the subject also receives a standard of care for CVD or risk management thereof. The present disclosure also provides methods of reducing CRP levels in a subject comprising administering an amount of astaxanthin to the subject, as described herein, wherein the subject is at risk for or suffers from CVD. Additional related methods of altering levels of one or more of: TNF-α, IL-1β, IL-6, INF-γ, oxidized LDL, total cholesterol, LDL cholesterol, VLDL cholesterol, triglycerides, FOXO3 activation, and HDL cholesterol, comprising administering astaxanthin to the subject are further provided herein.

### CROSS-REFERENCE TO RELATED APPLICATIONS

This application claims priority to U.S. Provisional Application No. 62/729,152, filed on Sep. 10, 2018, the entire contents of which is incorporated by reference herein.

### BACKGROUND

Coronary artery disease is the number one cause of mortality in the United States and many developed countries. The process of atherosclerosis begins in childhood and generally progresses with age. The rate and extent of progression vary per individual. Well-recognized conventional risk factors that increase the rate of progression include cigarette smoking, Diabetes Mellitus, hypertension, hyperlipidemia, and a family history of cardiovascular disease. Males are generally affected at a younger age than females (“Screening for asymptomatic coronary artery disease. U.S. Preventive Services Task Force.”, Am Fam Physician. 1989 December; 40(6):99-104).

Inflammation has been recognized as a common mechanism for all of these risk factors, and as an independent marker, playing a key role in the development of atherosclerosis from the early stages through development of atherosclerotic plaques, instability, and rupture. A host of inflammatory health biomarkers are linked with this process, including CRP, TNF-α, IL-1β, IL-6, and oxLDL. The JUPITER (Justification for the Use of Statins in Prevention, an Intervention Trial Evaluating Rosuvastatin) trial demonstrated that elevated CRP>2.0 mg/L is an independent risk factor for coronary events, even in subjects with LDL-C<130 mg/dL, and that reduction in CRP improved cardiac outcomes. The JUPITER trial randomized subjects to 20 mg/D of rosuvastatin vs. placebo. At enrollment, the median CRP was 4.2 mg/L, and patients who achieved a reduction in CRP<2.0 had a 62% reduction in vascular events. Published in 2009, the JUPITER trial results had a positive effect on the practice of clinical medicine, with more attention to CRP as a risk factor for cardiac events and broader use of statins (Ridker, P M, Circ Cardiovas Qual Outcomes. 2009; 2:279-285).

Because rosuvastatin lowers LDL-C as well as CRP, the medical community designed two large, multi-center, prospective, placebo controlled studies to further clarify whether reduction of inflammation reduces cardiac events in the absence of any effects on lipids.

Results were published in August 2017 from the CANTOS (Canakinumab ANti-inflammatory Thrombosis Outcome Study) trial (Ridker, P M, et al, N Engl J Med. 2017 Sep. 21; 377(12):1119-1131), comparing treatment with canakinumab vs. placebo for 48 months. Canakinumab is a monoclonal antibody that targets IL-113, resulting in a reduction of IL-6 and CRP, but has no effect on LDL-C. This study enrolled 10,061 patients at multiple centers in 39 countries. Study patients had a history of previous myocardial infarction and CRP>2.0 mg/L, and were generally treated with standard of care therapy for hypertension, diabetes, hyperlipidemia, and anti-ischemics. Patients were randomized to treatment with placebo, 50 mg, 150 mg, or 300 mg of canakinumab injections every 3 months and followed for 48 months.

Data analysis showed that patients with active treatment noted a reduction in CRP and IL-6, with no change in lipids. The reduction in CRP noted at three months was generally maintained in the same range throughout the trial. The 150 mg treatment group had a 15% reduction in recurrent myocardial infarctions. Unfortunately, the active treatment groups had more deaths from infection (mostly tuberculosis) hence there was no net reduction in all-cause mortality. The results of the CANTOS trial are best understood by evaluating the response to therapy rather than the dose. Patients with CRP>2.0 mg/L (regardless of dose) at three months had results very similar to placebo recipients. Subjects with a reduction of CRP<2.0 mg/L (regardless of dose) had a 25% reduction in the primary end point (a composite of myocardial infarction, stroke, or cardiovascular death) and had a 31% reduction in cardiovascular death and all-cause mortality. These results were highly statistically significant. To further demonstrate the benefits of reducing inflammation, the best results were in patients with CRP<1.2 mg/L post-treatment.

Following publication of the results in August 2017, the response to the CANTOS trial was summarized as “one important step for clinical cardiology but a giant leap for vascular biology.” (Baylis R A et al, Arterioscler Thromb Vasc Biol. 2017 November; 37(11):e174-e177). The “giant leap” is documented proof for the “inflammatory hypothesis,” by demonstrating that patients with cardiac disease benefit from reduction of inflammation, even without any additional reduction in lipids, while being treated aggressively by conventional standard of care therapy. The “one important step” is a cautious assessment that canakinumab is unlikely to be widely used in a clinical setting, mostly due to the cost (currently about $16,000 per injection), the variable response, and concerns about the overall risk-to-benefit ratio. Reviewers also noted that canakinumab is a very narrow intervention, effecting only one target, and that other interventions with broader anti-inflammatory action should be explored.

A second large, multi-center, prospective, randomized, placebo controlled study was initiated about the same time as the CANTOS trial to evaluate the “inflammatory hypothesis.” The CIRT (Cardiovascular Inflammation Reduction Trial) trial (Ridker et al., N Engl J Med 2019 February; 380: 752-762) initially planned to enroll 7,000 patients at 565 centers in the US and Canada, but the study was terminated for futility in April 2018 after enrollment of 4,786 patients at 417 centers. The population was somewhat broader than the CANTOS study, and included patients with CRP>2.0 mg/L and proven multi-vessel coronary artery disease plus either Diabetes Mellitus, Type II or metabolic syndrome as well as patients with a history of myocardial infarction. Patients were randomized to methotrexate at 15-20 mg/week vs. placebo. The results of the CRT trial demonstrated that administration of methotrexate did not reduce levels of IL-1β, IL-6, or CRP and did not result in fewer cardiovascular events compared to placebo, providing further evidence of the importance of this pathway in the reduction of cardiovascular events. While this dose was considered to have a reasonable risk-to-benefit ratio in these patients, long term use is also associated with substantial toxicities including hepatic fibrosis or cirrhosis, myelosuppression, pulmonary damage, oral ulcers, and nausea, vomiting, or diarrhea.

There is a need for a safe, effective agent with broad mechanisms of action for maintenance of inflammatory health in subjects with cardiovascular risk factors, which can be used with conventional treatments without negative drug-study product interactions, and if it can also benefit lipid, metabolic, liver, and joint health.

### SUMMARY

The present disclosure provides methods of preventing or treating cardiovascular disease (CVD) in a subject. In exemplary embodiments, the methods prevent or treat CVD by reducing C-reactive protein (CRP) levels. In exemplary embodiments, the method comprises administering a therapeutically effective amount of astaxanthin to the subject. In some embodiments, the subject is one who also receives a standard of care for CVD or CVD risk management.

The present disclosure also provides methods of supporting cardiovascular health in a subject at risk for or suffering from CVD. In exemplary embodiments, the methods support cardiovascular health by reducing CRP levels. In exemplary embodiments, the method comprises administering astaxanthin, e.g., a therapeutically effective amount of astaxanthin, to the subject. In some embodiments, the subject is one who also receives a standard of care for CVD or CVD risk management.

Further provided are methods of supporting inflammatory health in a subject at risk for or suffering from CVD. In exemplary embodiments, the methods prevent or treat CVD by reducing CRP levels. In exemplary embodiments, the method comprises administering astaxanthin, e.g., a therapeutically effective amount of astaxanthin, to the subject. In some embodiments, the subject is one who also receives a standard of care for CVD or CVD risk management.

Also provided by the present disclosure are methods of reducing CRP in a subject. In exemplary embodiments, the method comprises administering astaxanthin at an amount of about 24 mg/day or about 12 mg twice a day. In alternative or additional embodiments, the method comprises administering astaxanthin at a total daily amount of about 96 mg/day or about 48 mg twice a day. In alternative or additional embodiments, the method comprises administering astaxanthin at an amount that results in a level of astaxanthin in the bloodstream in the range of about 0.1 to about 1 μg/mL in plasma or serum. In alternative or additional embodiments, the method comprises administering astaxanthin at an amount that results in a level of astaxanthin in the bloodstream in the range of about 1 to about 10 μg/mL in plasma or serum. In exemplary embodiments of such methods of reducing CRP in a subject, the subject is at risk for or suffers from CVD.

The present disclosure additionally provides methods of modifying levels of inflammatory health biomarkers or cardiovascular health biomarkers in a subject at risk for or suffering from CVD comprising administering astaxanthin to the subject. In exemplary embodiments, the method (a) reduces levels of one or more of the following: TNF-α, IL-1β, IL-6, INF-γ, oxidized LDL, total cholesterol, LDL cholesterol, VLDL cholesterol, triglycerides, HbA1c, ALT, AST, body weight, or blood pressure; and/or (b) increases levels of one or more of the following: FOXO3 activation or HDL cholesterol. In exemplary embodiments of such methods of modifying levels of biomarkers in a subject, the subject also receives standard of care for CVD or CVD risk management. In some cases, the method does not increase a subject's liver enzymes, e.g., ALT and/or AST.

In alternative or additional embodiments, the method of modifying levels of inflammatory biomarkers in a subject comprises administering astaxanthin at an amount of about 24 mg/day, e.g. about 12 mg twice a day. In alternative or additional embodiments, the method comprises administering astaxanthin at an amount of about 96 mg/day, e.g., about 48 mg twice a day. In exemplary embodiments, the subject is at risk for or suffers from CVD. In additional or alternative embodiments, the method comprises administering astaxanthin at an amount that results in a level of astaxanthin in the bloodstream in the range of about 0.1 to about 1 μg/mL in plasma or serum. In additional or alternative embodiments, the method comprises administering astaxanthin at an amount that results in a level of astaxanthin in the bloodstream in the range of 1 to 10 μg/mL in plasma or serum. In exemplary embodiments of such methods of modifying biomarker levels, the subject is at risk for or suffers from CVD.

### BRIEF DESCRIPTION OF THE DRAWINGS

FIG. 1 is a synopsis table for the CHASE clinical trial.

FIG. 2 is a table listing the schedule of activities of the CHASE clinical trial.

FIG. 3 represents a table (Table 3) referenced in the Examples.

FIGS. 4a and 4b are a pair of sample calculations.

FIGS. 5 and 6 provide additional interim data from the CHASE trial. FIG. 5 is a scatterplot of hs CRP (mg/L) plotted as a function of astaxanthin concentration (ng/mL) at V6 and FIG. 6 is a scatterplot of % change in triglycerides (V2 to V6) plotted as a function of astaxanthin concentration (ng/mL).

### DETAILED DESCRIPTION

The present disclosure provides methods comprising administering to a subject astaxanthin. Astaxanthin is a nutrient primarily present in seafood and has attracted attention due to its unique chemistry and wide-ranging potential clinical benefits. Astaxanthin neutralizes free radicals without being destroyed in the process. Astaxanthin has a unique linear, polar-nonpolar-polar molecular structure and size that equips it to precisely insert into the cellular membrane and span its entire width, and has been shown to accumulate in the mitochondria (Kidd P., Altern Med Rev. 2011 December; 16(4): 355-64). Astaxanthin is one of the most potent quenchers of free radicals and reactive oxygen and nitrogen species known: it is 11 times more potent than beta-carotene and 550 times greater than alpha-tocopherol (Fassett R B, Coombes J S, Molecules. 2012; 17: 2030-48; Shimidzu N., Fish Sci. 1996; 62: 134-7; Krinsky N I., Free Radic Biol Med. 1989; 7: 617-35; and Miki W., Pure Appl. Chem. 1991; 63: 141-6).

Humans do not make astaxanthin. In nature, astaxanthin is produced by algae, bacteria, and fungi, and concentrated up the food chain in krill, crabs, lobsters, shrimp, salmon, and trout, not to mention whales, bears, and humans at the top of the food chain. Astaxanthin is generally present in nature as a mixture of esters with various fatty-acids esterified to one or both polar ends of the astaxanthin molecule, or in its free non-esterified form. Astaxanthin has two chiral sites and occurs in nature as all three stereo isomeric forms: the enantiomers S,S′, R,R′, and the symmetrical meso form. Astaxanthin also occurs in nature as several geometric isomers including all-trans and various cis forms.

The most common natural source of astaxanthin is the single-celled alga Haematococcus pluvialis (H. pluvialis). Cultures of this alga historically comprised the largest commercial source of astaxanthin supplements. Astaxanthin supplements from several microalgal sources have been designated as generally recognized as safe (GRAS) in accordance with FDA regulations and are widely sold in capsules ranging from 4 mg to 12 mg, either alone or in combination with other nutrients.

Microalgal based production of astaxanthin has certain environmental requirements, technical complexities and capacity limitations, as well as inherent molecular diversity in the naturally occurring esters. For these reasons there is interest in synthetic production of a pure chemical entity under current Good Manufacturing Practice (GMP) standards.

Astaxanthin is a xanthophyll carotenoid. In pure, non-esterified form, astaxanthin has a molecular weight of approximately 596.84 g/mol. The molecular formula is C₄₀H₅₂O₄. Astaxanthin has a complex three-dimensional chemistry and exists naturally as trans and cis isomers. Below is a diagram of all-trans astaxanthin.

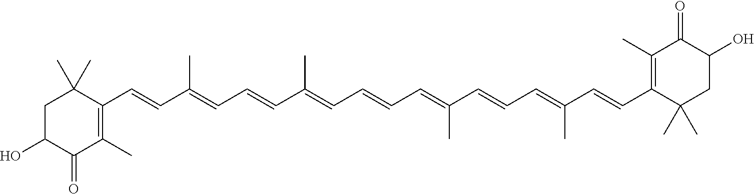 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

In exemplary aspects, the astaxanthin used in the methods of the present disclosure comprises the structure above. In exemplary aspects, the astaxanthin used in the methods of the present disclosure has the structure which is identical to naturally-occurring astaxanthin. In exemplary aspects, the astaxanthin used in the methods is non-esterified. In exemplary aspects, the astaxanthin is formulated as microbeadlets. In exemplary instances, the astaxanthin beadlet formulation used in ZanthoSyn® which has a GRAS designation in accordance with FDA regulations. This formulation was shown in non-clinical studies to have superior bioavailability to microalgal astaxanthin, which is a non-homogeneous mix of astaxanthin in various esterified forms. The safety, tolerability, and pharmacokinetics of the astaxanthin in various forms from various sources has been demonstrated in prior clinical studies^{27-29, 31-33, 35, 38-57}.

In exemplary aspects of the methods of the present disclosure the astaxanthin is

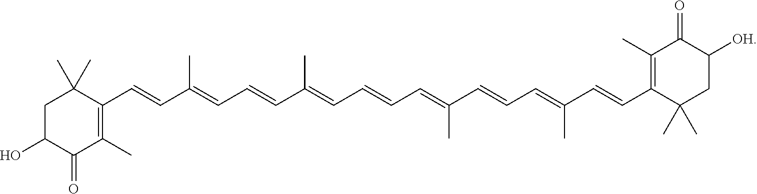 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

In some aspects, the astaxanthin is a mixture of (S,S′), (R,R′), and meso isomer forms of astaxanthin. In certain instances, the astaxanthin is synthetically produced.

Pharmaceutical Compositions

In exemplary aspects of the present disclosure, the astaxanthin is part of a pharmaceutical composition when administered to the subject. The pharmaceutical composition comprising astaxanthin in most aspects is purified and sterile. The term “purified” as used herein means having been increased in purity, wherein “purity” is a relative term, and not to be necessarily construed as absolute purity. In exemplary aspects, the purity of the compound (e.g., in the composition) is at least or about 50%, at least or about 60%, at least or about 70%, at least or about 80%, at least or about 90%, at least or about 95%, or at least or about 98% or is about 100%. In exemplary aspects, the pharmaceutical composition comprises a pharmaceutically acceptable carrier. As used herein, the term “pharmaceutically acceptable carrier” includes any of the standard pharmaceutical carriers, such as a phosphate buffered saline solution, water, oil, emulsions such as an oil/water or water/oil emulsion, and various types of wetting agents. The term also encompasses any of the agents approved by a regulatory agency of the US Federal government or listed in the US Pharmacopeia for use in animals, including humans. The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In exemplary aspects, the pharmaceutical composition comprises formulation materials that are nontoxic to recipients at the dosages and concentrations employed. In specific embodiments, pharmaceutical compositions comprising an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as bcnzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbatc, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In exemplary embodiments, the pH of the pharmaceutical composition is between 5.5 and 7.5.

In exemplary aspects, the pharmaceutical composition comprises free-flowing particles or beadlets (e.g., microbeadlets) comprising astaxanthin. In exemplary aspects, the pharmaceutical composition comprises a gelatin capsule or a vegetable-based capsule (e.g., a tapioca capsule or a vegetable cellulose capsule) comprising free-flowing particles or beadlets (e.g., microbeadlets) comprising astaxanthin. In exemplary aspects, the pharmaceutical composition additionally comprises one or more antioxidants. In exemplary instances, the pharmaceutical composition additionally comprises one or more of: modified food starch, corn starch, glucose syrup, sodium ascorbate, and DL-alpha-tocopherol. In some instances, astaxanthin is dispersed in a corn-starch coated matrix of modified food starch and glucose syrup with sodium ascorbate and DL-alpha-tocopherol. In exemplary aspects, the pharmaceutical composition comprises a gelatin capsule or a vegetable-based capsule comprising free-flowing particles or beadlets (e.g., microbeadlets) comprising astaxanthin dispersed in a corn-starch coated matrix of modified food starch and glucose syrup with sodium ascorbate and DL-alpha-tocopherol.

In exemplary aspects, the pharmaceutical composition comprises: (a) astaxanthin, modified food starch, corn starch, glucose syrup, sodium ascorbate, DL-alpha-tocopherol, microcrystalline cellulose, tapioca capsule; (b) astaxanthin, modified food starch, corn starch, glucose syrup, sodium ascorbate, DL-alpha-tocopherol, microcrystalline cellulose, vegetable stearate, vegetable cellulose capsule; (c) astaxanthin, modified food starch, corn starch, glucose syrup, sodium ascorbate, DL-alpha-tocopherol, microcrystalline cellulose, vegetable stearate, vegetable cellulose capsule, titanium dioxide; (d) astaxanthin, modified food starch, corn starch, glucose syrup, sodium ascorbate, DL-alpha-tocopherol, microcrystalline cellulose, vegetable stearate, gelatin capsule, FD&C blue #1, titanium dioxide.

### Routes of Administration, Dosages and Regimen

With regard to the present disclosure, the astaxanthin, or pharmaceutical composition comprising the same, can be administered to the subject via any suitable route of administration. For example, the active agent can be administered to a subject via parenteral, nasal, oral, pulmonary, topical, vaginal, ocular, or rectal administration. In exemplary embodiments, the astaxanthin, or pharmaceutical composition comprising the same, is administered orally. The following discussion on routes of administration is merely provided to illustrate exemplary embodiments and should not be construed as limiting the scope in any way.

Formulations suitable for oral administration can consist of (a) liquid solutions, such as an effective amount of astaxanthin dissolved or dispersed in diluents, such as water, saline, oil, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the astaxanthin in a flavor, usually sucrose and acacia or tragacanth, as well as pastilles comprising the astaxanthin in an inert base, such as gelatin and glycerin, or sucrose and acacia, emulsions, gels, and the like containing, in addition to, such excipients as are known in the art.

In exemplary embodiments, the astaxanthin is administered orally to the subject. In exemplary aspects, the astaxanthin is a tablet or capsule or caplet.

The pharmaceutical composition in exemplary aspects is modified to have any type of in vivo release profile. In some aspects, the pharmaceutical composition is an immediate release, controlled release, sustained release, extended release, delayed release, or bi-phasic release formulation. Methods of formulating peptides for controlled release are known in the art. See, for example, Qian et al., J Pharm 374: 46-52 (2009) and International Patent Application Publication Nos. WO 2008/130158, WO2004/033036; WO2000/032218; and WO 1999/040942.

The instant compositions may further comprise, for example, micelles or liposomes, or some other encapsulated form, or may be administered in an extended release form to provide a prolonged storage and/or delivery effect.

The disclosed pharmaceutical composition may be administered according to any regimen including, for example, daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly.

In some aspects, astaxanthin, or the pharmaceutical composition comprising the same, is taken on an empty stomach or after a period of fasting. In alternative aspects, the astaxanthin is administered with food, or within about 30 minutes after food (e.g., a meal).

Astaxanthin is useful in methods of reducing CRP and modifying the levels of inflammatory biomarkers, as described herein, and is thus believed to be useful in methods of treating or preventing a CVD, supporting cardiovascular health, and supporting inflammatory health. For purposes of the disclosure, the amount or dose of astaxanthin administered should be sufficient to effect, e.g., a therapeutic or prophylactic response, in the subject or animal over a reasonable time frame. For example, the dose of astaxanthin should be sufficient to treat a CVD by way of reducing CRP levels as described herein in a period of from about 1 to 4 minutes, 1 to 4 hours or 1 to 4 weeks or longer, e.g., 5 to 20 or more weeks, from the time of administration. In certain embodiments, the time period could be even longer. In some aspects, the effect is demonstrated within 1 to 2 weeks, within 4 weeks, within 8 weeks, within 12 weeks, within 24 weeks, within 48 weeks, or within 60 weeks.

The dose will be determined by the efficacy of the particular formulation and the condition of the animal (e.g., human), as well as the body weight of the animal (e.g., human) to be treated.

The dose of astaxanthin of the present disclosure also will be determined by the existence, nature and extent of any adverse side effects that might accompany the administration of a particular active agent of the present disclosure. Typically, the attending physician will decide the dosage of the active agent of the present disclosure with which to treat each individual patient, taking into consideration a variety of factors, such as age, body weight, general health, diet, sex, active agent of the present disclosure to be administered, route of administration, and the severity of the condition being treated. By way of example and not intending to limit the present disclosure, the dose of the active agent of the present disclosure can be about 0.0001 to about 1 g/kg body weight of the subject being treated/day, from about 0.0001 to about 0.001 g/kg body weight/day, or about 0.01 mg to about 1 g/kg body weight/day.

In exemplary aspects, the dose of astaxanthin is about 10 mg to about 40 mg, e.g., about 15 mg to about 40 mg, about 20 mg to about 40 mg, about 25 mg to about 40 mg, about 30 mg to about 40 mg, about 35 mg to about 40 mg, about 10 mg to about 35 mg, about 10 mg to about 30 mg, about 10 mg to about 25 mg, about 10 mg to about 20 mg, about 10 mg to about 15 mg. In exemplary aspects, the daily dose of astaxanthin is about 20 mg to about 30 mg, e.g., about 21 mg, about 22 mg, about 23 mg, about 24 mg, about 25, mg, about 26 mg, about 27 mg, about 28 mg, about 29 mg, or about 30 mg. Alternatively, the daily dose is about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 75 mg to about 80 mg, about 80 mg to about 115 mg, about 85 mg to about 115 mg, about 90 mg to about 115 mg, about 95 mg to about 115 mg, about 100 mg to about 115 mg, about 105 mg to about 115 mg, about 110 mg to about 115 mg. In exemplary aspects, the daily dose is about is about 90 mg to about 100 mg, e.g., about 91 mg, about 92 mg, about 93 mg, about 94 mg, about 95 mg, about 96 mg, about 97 mg, about 98 mg, about 99 mg, about 100 mg.

In exemplary aspects, the dose of astaxanthin administered to the subject is a twice daily dose. In exemplary aspects, the twice daily dose is about 6 mg to about 20 mg, e.g., about 8 mg to about 20 mg, about 10 mg to about 20 mg, about 12 mg to about 20 mg, about 14 mg to about 20 mg, about 16 mg to about 20 mg, about 18 mg to about 20 mg, about 6 mg to about 18 mg, about 6 mg to about 16 mg, about 6 mg to about 14 mg, about 6 mg to about 12 mg, about 6 mg to about 10 mg, or about 6 mg to about 8 mg. In exemplary aspects, the twice daily dose is about 10 mg to about 15 mg, e.g., about 11 mg, about 12 mg, about 13 mg, about 14 mg. Alternatively, the twice daily dose is about 30 mg to about 60 mg, e.g., about 35 mg to about 60 mg, about 40 mg to about 60 mg, about 45 mg to about 60 mg, about 50 mg to about 60 mg, about 55 mg to about 60 mg, about 30 mg to about 55 mg, about 30 mg to about 50 mg, about 30 mg to about 45 mg, about 30 mg to about 40 mg, about 30 mg to about 35 mg. In exemplary aspects, the twice daily dose is about 40 mg to about 50 mg, e.g., about 41 mg, about 42 mg, about 43 mg, about 44 mg, about 45 mg, about 46 mg, about 47 mg, about 48 mg, about 49 mg, about 50 mg.

In alternative or additional aspects, the amount of astaxanthin administered results in a level of astaxanthin in the bloodstream in the range of 0.1 to 1 μg/mL in plasma or serum. In exemplary aspects, the astaxanthin is administered to achieve a plasma or serum level of about 0.1 μg astaxanthin/mL plasma or serum to about 0.9 μg astaxanthin/mL plasma or serum, about 0.1 μg astaxanthin/mL plasma or serum to about 0.8 μg astaxanthin/mL plasma or serum, about 0.1 μg astaxanthin/mL plasma or serum to about 0.7 astaxanthin/mL plasma or serum, about 0.1 μg astaxanthin/mL plasma or serum to about 0.6 μg astaxanthin/mL plasma or serum, about 0.1 μg astaxanthin/mL plasma or serum to about 0.5 μg astaxanthin/mL plasma or serum, about 0.1 μg astaxanthin/mL plasma or serum to about 0.4 μg astaxanthin/mL plasma or serum, about 0.1 μg astaxanthin/mL plasma or serum to about 0.3 μg astaxanthin/mL plasma or serum, about 0.1 μg astaxanthin/mL plasma or serum to about 0.2 μg astaxanthin/mL plasma or serum, about 0.2 μg astaxanthin/mL plasma or serum to about 1.0 μg astaxanthin/mL plasma or serum, about 0.3 μg astaxanthin/mL plasma or serum to about 1.0 μg astaxanthin/mL plasma or serum, about 0.4 μg astaxanthin/mL plasma or serum to about 1.0 μg astaxanthin/mL plasma or serum, about 0.5 μg astaxanthin/mL plasma or serum to about 1.0 μg astaxanthin/mL plasma or serum, about 0.6 μg astaxanthin/mL plasma or serum to about 1.0 μg astaxanthin/mL plasma or serum, about 0.7 μg astaxanthin/mL plasma or serum to about 1.0 μg astaxanthin/mL plasma or serum, about 0.8 μg astaxanthin/mL plasma or serum to about 1.0 μg astaxanthin/mL plasma or serum, about 0.9 μg astaxanthin/mL plasma or serum to about 1.0 μg astaxanthin/mL plasma or serum.

In some aspects, the amount of astaxanthin administered results in a level of astaxanthin in the bloodstream in the range of 1 to 10 μg/mL in plasma or serum. In exemplary aspects, the astaxanthin is administered to achieve a plasma or serum level of about 1 μg astaxanthin/mL plasma or serum to about 9 μg astaxanthin/mL plasma or serum, about 1 μg astaxanthin/mL plasma or serum to about 8 μg astaxanthin/mL plasma or serum, about 1 μg astaxanthin/mL plasma or serum to about 7 μg astaxanthin/mL plasma or serum, about 1 μg astaxanthin/mL plasma or serum to about 6 μg astaxanthin/mL plasma or serum, about 1 μg astaxanthin/mL plasma or serum to about 5 μg astaxanthin/mL plasma or serum, about 1 μg astaxanthin/mL plasma or serum to about 4 μg astaxanthin/mL plasma or serum, about 1 μg astaxanthin/mL plasma or serum to about 3 μg astaxanthin/mL plasma or serum, about 1 μg astaxanthin/mL plasma or serum to about 2 μg astaxanthin/mL plasma or serum, about 2 μg astaxanthin/mL plasma or serum to about 10 μg astaxanthin/mL plasma or serum, about 3 μg astaxanthin/mL plasma or serum to about 10 μg astaxanthin/mL plasma or serum, about 4 μg astaxanthin/mL plasma or serum to about 10 μg astaxanthin/mL plasma or serum, about 5 μg astaxanthin/mL plasma or serum to about 10 μg astaxanthin/mL plasma or serum, about 6 μg astaxanthin/mL plasma or serum to about 10 μg astaxanthin/mL plasma or serum, about 7 μg astaxanthin/mL plasma or serum to about 10 μg astaxanthin/mL plasma or serum, about 8 μg astaxanthin/mL plasma or serum to about 10 μg astaxanthin/mL plasma or serum, about 9 μg astaxanthin/mL plasma or serum to about 10 μg astaxanthin/mL plasma or serum.

### Combinations

In some embodiments, the astaxanthin is administered alone, and in alternative embodiments, astaxanthin is administered in combination with another therapeutic agent. In some aspects, the other therapeutic agent aims to treat or prevent a cardiovascular disease. In exemplary aspects, the other agent is a statin, a hypertension medication, niacin, fibrate, cholesterylester transfer protein (CETP) inhibitor, an anti-diabetic agent, or a vasoactive agent. In some cases, the other agent is fenofibrate or omega-3-acid ethyl esters (lovaza). The statin in some aspects is atorvastatin, fluvastatin, lovastatin, pravastatin, simvastatin, or rosuvastatin. The hypertension medication may be any of those described herein. The CETP inhibitor may be, e.g., torcetrapib, dalcetrapib, evacetrapib, obicetrapib, or anacetrapib. The anti-diabetic agent may be any of those described herein. The vasoactive agent may be an inotrope which increases myocardial contractility, a vasopressor which causes vasoconstriction leading to increased systemic and/or pulmonary vascular resistance, an inodilator which causes vasodilation leading to decreased systemic and/or pulmonary vascular resistance. The inotrope may be, e.g., adrenaline, dobutamine, isoprenaline, and ephedrine. In some aspects, the vasopressor is noradrenaline, vasopressin, metaraminol, or methylene blue. In certain instances, the inodilator is milrinone or levosimendan. In exemplary instances, the vasoactive agent is dopamine. In some aspects, the vasoactive agent is a catecholamine, e.g., adrenaline, noradrenaline, phenylephedrine and metaraminol, ephedrine, dopamine, dobutamine, isoprenaline. In some aspects, the vasoactive agent is levosimendan or milrinone.

In exemplary aspects, the astaxanthin is administered in combination with a hypertension medication, including, but not limited to, a diuretic, a beta blocker, an ACE inhibitor, a angiotensin receptor blocker, a calcium channel blocker, an alpha blocker, an alpha-2 receptor agonist, a central agonist, a renin inhibitor, an arterial vasodilator, and the like. In some aspects, the diuretic is any drug that increases urination to reduce sodium and fluid in the body and optionally is selected from the group consisting of: bumetanide, chlorthalidone, chlorothiazide, ethacrynate, furosemide, hydrochlorothiazide HCTZ (Esidrix, hydrodiuril, microzide), indapamide, methclothiazide, metolazone, torsemide, amilioride, spironolactone, triamterene, amiloride hydrochloride. In exemplary aspects, the beta blocker is any that act directly on the heart to reduce heart rate, force of pumping, and blood volume. In some aspects, the beta blocker is acebutolol, atenolol, bisoprolol fumarate, carvedilol, esmilol, labetalol, metoprolol tartrate, metoprolol succinate, nadolol, nebivolol, penbutolol sulfate, propranolol, or sotalol. In some aspects, the ace inhibitor is benazepril hydrochloride, captopril, enalapril maleate, fosinopril sodium, lisinopril, moexipril, perindopril, quinapril, ramipril, or trandolapril. In some instances, the angiotensin II receptor blocker is azilsartan, candesartan, eprosartan mesylate, irbesartan, losartan potassium, olmesarten, telmisartan, valsartan. In some instances, the calcium channel blocker is amlodipine besylate, clevidipine, diltiazem hydrochloride, felodipine, isradipine, nicardipine, nifedipene, nimodipine, nisoldipine, verapamil hydrochloride. In some regards, the alpha blocker is doxazosin mesylate, prazosin hydrochloride, or terazosin hydrochloride. In certain aspects, the alpha-2 receptor agonist is methyldopa. In certain aspects, the central agonist is clonidine hydrochloride and guanfacine hydrochloride. In some instances, the hypertension medication is a peripheral adrenergic inhibitor, such as hyanadrel, guanethidine monosulfate, or reserpine. In certain instances, the vasodilator is minoxidil or hydralazine. In various embodiments, the hypertension agent is amlodipine, amlodipine/benazepril, amlodipine/valsartan, atenolol, carvedilol, cortisode, entresto, furosemide, hydrochlorothiazide (HCTZ), labetalol, lisinopril, Lisinopril/HCTZ, losartan, losartan/HCTZ, metoprolol, Ramipril, or triamterene/HCTZ, or any combination thereof.

In exemplary aspects, the astaxanthin is administered in combination with an anti-diabetic or anti-obesity agent. Anti-diabetic agents known in the art or under investigation include insulin, leptin, Peptide YY (PYY), Pancreatic Peptide (PP), fibroblast growth factor 21 (FGF21), Y2Y4 receptor agonists, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARy inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; glucokinase activators (GKA); glucagon receptor antagonists (GRA); or FBPase (fructose 1,6-bisphosphatase) inhibitors. Anti-obesity agents known in the art or under investigation include appetite suppressants, including phenethylamine type stimulants, phentermine (optionally with fenfluramine or dexfenfluramine), diethylpropion (Tenuate®), phendimetrazine (Prelu-2®, Bontril®), benzphetamine (Didrex®), sibutramine (Meridia®, Reductil®); rimonabant (Acomplia®), other cannabinoid receptor antagonists; oxyntomodulin; fluoxetine hydrochloride (Prozac); Qnexa (topiramate and phentermine), Excalia (bupropion and zonisamide) or Contrave (bupropion and naltrexone); or lipase inhibitors, similar to XENICAL (Orlistat) or Cetilistat (also known as ATL-962), or GT 389-255.

In exemplary aspects, the astaxanthin is administered in combination with an agent for treatment of non-alcoholic fatty liver disease or NASH. Agents used to treat non-alcoholic fatty liver disease include ursodeoxycholic acid (a.k.a., Actigall, URSO, and Ursodiol), Metformin (Glucophage), rosiglitazone (Avandia), Clofibrate, Gemfibrozil, Polymixin B, and Betaine.

Cardiovascular Diseases and Cardiovascular Health

The present disclosure provides methods of preventing or treating cardiovascular disease (CVD) in a subject. In exemplary embodiments, the methods prevent or treat CVD by reducing C-reactive protein (CRP) levels. In exemplary embodiments, the method comprises administering a therapeutically effective amount of astaxanthin to the subject. In some embodiments, the subject is one who also receives a standard of care for CVD or CVD risk management.

The present disclosure also provides methods of supporting cardiovascular health in a subject at risk for or suffering from CVD. In exemplary embodiments, the methods support cardiovascular health by reducing CRP levels. In exemplary embodiments, the method comprises administering astaxanthin to the subject. In some embodiments, the subject is one who also receives a standard of care for CVD or CVD risk management. As used herein, the term “cardiovascular health” refers to the healthy structure or function of the heart and blood vessels.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating CVD of the present disclosure can provide any amount or any level of treatment. Furthermore, the treatment provided by the method of the present disclosure can include treatment of one or more conditions or symptoms or signs of the CVD being treated. Also, the treatment provided by the methods of the present disclosure can encompass slowing the progression of the CVD. For example, the methods can treat CVD by virtue of reducing build-up of plaque on the artery walls, treating angina, dyspnea, or nausea, managing CVD or signs or symptoms thereof, and the like. As used herein, the term “prevent” as well as word related thereto refers to delaying the onset or recurrence of the CVD by at least 1 day, 2 days, 4 days, 6 days, 8 days, 10 days, 15 days, 30 days, two months, 3 months, 4 months, 6 months, 1 year, 2 years, 3 years, 4 years, or more. In exemplary aspects, the methods treat or prevent by way increasing the survival of the subject.

As used herein, the term “cardiovascular disease” or “CVD” refers to a class of diseases involving the heart or blood vessels. CVD includes coronary artery diseases (CAD), e.g., as angina and myocardial infarction (commonly known as a heart attack), as well, as stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis. In some aspects, the CVD includes angina, arrhythmia, congenital heart disease, CAD, dilated cardiomyopathy, heart attack, heart failure, hypertrophic cardiomyopathy, mitral regurgitation, mitral valve prolapse, pulmonary stenosis, rheumatic heart disease. The CVD in exemplary aspects, involves the blood vessels and thus may be a vascular disease. Such vascular diseases include, but are not limited to: coronary artery disease, peripheral arterial disease, cerebrovascular disease, renal artery stenosis, aortic aneurysm, Raynaud's disase, Buerger's disease, peripheral venous disease, atherosclerosis, stroke, venous blood clots, and blood clotting disorders. The CVD in exemplary aspects, involves the heart and may be one of the following: cardiomyopathy, hypertensive heart disease, heart failure, pulmonary heart disease, cardiac dysrhythmias, an inflammatory heart disease (e.g., endocarditis, inflammatory cardiomegaly, myocarditis, eosinophilic myocarditis), a valvular heart disease, congenital heart disease, or rheumatic heart disease.

As used herein, the term “standard of care for CVD or CVD risk management” refers to any of the following agents which aims to treat or prevent a cardiovascular disease: a nitrate, a statin, a hypertension medication, niacin, fibrate, CETP inhibitor, an anti-diabetic agent, a vasoactive agent. In some aspects, the standard of care for CVD or CVD risk management is a nitrate or other anti-anginal therapeutic agent (e.g., a beta blocker, a calcium channel blocker). Such agents are described herein and also known in the art.

In exemplary aspects, the dose of astaxanthin is about 10 mg to about 40 mg, e.g., about 15 mg to about 40 mg, about 20 mg to about 40 mg, about 25 mg to about 40 mg, about 30 mg to about 40 mg, about 35 mg to about 40 mg, about 10 mg to about 35 mg, about 10 mg to about 30 mg, about 10 mg to about 25 mg, about 10 mg to about 20 mg, about 10 mg to about 15 mg. In exemplary aspects, the daily dose of astaxanthin is about 20 mg to about 30 mg, e.g., about 21 mg, about 22 mg, about 23 mg, about 24 mg, about 25, mg, about 26 mg, about 27 mg, about 28 mg, about 29 mg, or about 30 mg. Alternatively, the daily dose is about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 75 mg to about 80 mg, about 80 mg to about 115 mg, about 85 mg to about 115 mg, about 90 mg to about 115 mg, about 95 mg to about 115 mg, about 100 mg to about 115 mg, about 105 mg to about 115 mg, about 110 mg to about 115 mg. In exemplary aspects, the daily dose is about is about 90 mg to about 100 mg, e.g., about 91 mg, about 92 mg, about 93 mg, about 94 mg, about 95 mg, about 96 mg, about 97 mg, about 98 mg, about 99 mg, about 100 mg.

In exemplary aspects, the dose of astaxanthin administered to the subject is a twice daily dose. In exemplary aspects, the twice daily dose is about 6 mg to about 20 mg, e.g., about 8 mg to about 20 mg, about 10 mg to about 20 mg, about 12 mg to about 20 mg, about 14 mg to about 20 mg, about 16 mg to about 20 mg, about 18 mg to about 20 mg, about 6 mg to about 18 mg, about 6 mg to about 16 mg, about 6 mg to about 14 mg, about 6 mg to about 12 mg, about 6 mg to about 10 mg, or about 6 mg to about 8 mg. In exemplary aspects, the twice daily dose is about 10 mg to about 15 mg, e.g., about 11 mg, about 12 mg, about 13 mg, about 14 mg. Alternatively, the twice daily dose is about 30 mg to about 60 mg, e.g., about 35 mg to about 60 mg, about 40 mg to about 60 mg, about 45 mg to about 60 mg, about 50 mg to about 60 mg, about 55 mg to about 60 mg, about 30 mg to about 55 mg, about 30 mg to about 50 mg, about 30 mg to about 45 mg, about 30 mg to about 40 mg, about 30 mg to about 35 mg. In exemplary aspects, the twice daily dose is about 40 mg to about 50 mg, e.g., about 41 mg, about 42 mg, about 43 mg, about 44 mg, about 45 mg, about 46 mg, about 47 mg, about 48 mg, about 49 mg, about 50 mg.

In alternative or additional aspects, the amount of astaxanthin administered results in a level of astaxanthin in the bloodstream in the range of 0.1 to 1 μg/mL in plasma or serum. In exemplary aspects, the astaxanthin is administered to achieve a plasma or serum level of about 0.1 μg astaxanthin/mL plasma or serum to about 0.9 μg astaxanthin/mL plasma or serum, about 0.1 μg astaxanthin/mL plasma or serum to about 0.8 μg astaxanthin/mL plasma or serum, about 0.1 μg astaxanthin/mL plasma or serum to about 0.7 astaxanthin/mL plasma or serum, about 0.1 μg astaxanthin/mL plasma or serum to about 0.6 μg astaxanthin/mL plasma or serum, about 0.1 μg astaxanthin/mL plasma or serum to about 0.5 μg astaxanthin/mL plasma or serum, about 0.1 μg astaxanthin/mL plasma or serum to about 0.4 μg astaxanthin/mL plasma or serum, about 0.1 μg astaxanthin/mL plasma or serum to about 0.3 μg astaxanthin/mL plasma or serum, about 0.1 μg astaxanthin/mL plasma or serum to about 0.2 μg astaxanthin/mL plasma or serum, about 0.2 astaxanthin/mL plasma or serum to about 1.0 μg astaxanthin/mL plasma or serum, about 0.3 μg astaxanthin/mL plasma or serum to about 1.0 μg astaxanthin/mL plasma or serum, about 0.4 μg astaxanthin/mL plasma or serum to about 1.0 μg astaxanthin/mL plasma or serum, about 0.5 μg astaxanthin/mL plasma or serum to about 1.0 μg astaxanthin/mL plasma or serum, about 0.6 μg astaxanthin/mL plasma or serum to about 1.0 μg astaxanthin/mL plasma or serum, about 0.7 μg astaxanthin/mL plasma or serum to about 1.0 astaxanthin/mL plasma or serum, about 0.8 μg astaxanthin/mL plasma or serum to about 1.0 μg astaxanthin/mL plasma or serum, about 0.9 μg astaxanthin/mL plasma or serum to about 1.0 μg astaxanthin/mL plasma or serum.

In some aspects, the amount of astaxanthin administered results in a level of astaxanthin in the bloodstream in the range of 1 to 10 μg/mL in plasma or serum. In exemplary aspects, the astaxanthin is administered to achieve a plasma or serum level of about 1 μg astaxanthin/mL plasma or serum to about 9 μg astaxanthin/mL plasma or serum, about 1 μg astaxanthin/mL plasma or serum to about 8 μg astaxanthin/mL plasma or serum, about 1 μg astaxanthin/mL plasma or serum to about 7 μg astaxanthin/mL plasma or serum, about 1 μg astaxanthin/mL plasma or serum to about 6 μg astaxanthin/mL plasma or serum, about 1 μg astaxanthin/mL plasma or serum to about 5 μg astaxanthin/mL plasma or serum, about 1 μg astaxanthin/mL plasma or serum to about 4 μg astaxanthin/mL plasma or serum, about 1 μg astaxanthin/mL plasma or serum to about 3 μg astaxanthin/mL plasma or serum, about 1 μg astaxanthin/mL plasma or serum to about 2 μg astaxanthin/mL plasma or serum, about 2 μg astaxanthin/mL plasma or serum to about 10 μg astaxanthin/mL plasma or serum, about 3 μg astaxanthin/mL plasma or serum to about 10 μg astaxanthin/mL plasma or serum, about 4 μg astaxanthin/mL plasma or serum to about 10 μg astaxanthin/mL plasma or serum, about 5 μg astaxanthin/mL plasma or serum to about 10 μg astaxanthin/mL plasma or serum, about 6 μg astaxanthin/mL plasma or serum to about 10 astaxanthin/mL plasma or serum, about 7 μg astaxanthin/mL plasma or serum to about 10 μg astaxanthin/mL plasma or serum, about 8 μg astaxanthin/mL plasma or serum to about 10 μg astaxanthin/mL plasma or serum, about 9 μg astaxanthin/mL plasma or serum to about 10 μg astaxanthin/mL plasma or serum.

In exemplary aspects, the subject, prior to the administration of astaxanthin, exhibits a C-reactive protein (CRP) level of at least 2 mg/L, optionally, at least about 3 mg/L, at least about 4 mg/L, at least about 5 mg/L, at least about 6 mg/L, or more. In certain instances, the subject, following the administration of astaxanthin, exhibits a C-reactive protein (CRP) level below 2 mg/L, optionally, below about 1.9 mg/L, below about 1.8 mg/L, about 1.7 mg/L, about 1.6 mg/L, about 1.5 mg/L, about 1.4 mg/L, about 1.3 mg/L, about 1.2 mg/L, about 1.1 mg/L, about 1.0 mg/L, about 0.9 mg/L, about 0.8 mg/L, about 0.7 mg/L, about 0.6 mg/L, about 0.5 mg/L, about 0.4 mg/L, about 0.3 mg/L, about 0.2 mg/L, about 0.1 mg/L.

Methods of Reducing CRP

Also provided by the present disclosure are methods of reducing CRP in a subject. In exemplary embodiments, the method comprises administering astaxanthin at an amount of about 24 mg/day or about 12 mg twice a day. In alternative or additional embodiments, the method comprises administering astaxanthin at an amount of about 96 mg/day or about 48 mg twice a day. In alternative or additional embodiments, the method comprises administering astaxanthin at an amount that results in a therapeutic level of astaxanthin in the bloodstream in the range of about 0.1 to about 1 μg/mL in plasma or serum. In alternative or additional embodiments, the method comprises administering astaxanthin at an amount that results in a therapeutic level of astaxanthin in the bloodstream in the range of about 1 to about 10 μg/mL in plasma or serum. In exemplary embodiments of such methods of reducing CRP in a subject, the subject is at risk for or suffers from CVD.

As used herein, the phrase “reducing CRP levels” may refer to any decrease or reduction in CRP levels. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease. Methods of measuring CRP levels are known in the art and include the method described herein in Examples.

In exemplary aspects, the dose of astaxanthin is about 10 mg to about 40 mg, e.g., about 15 mg to about 40 mg, about 20 mg to about 40 mg, about 25 mg to about 40 mg, about 30 mg to about 40 mg, about 35 mg to about 40 mg, about 10 mg to about 35 mg, about 10 mg to about 30 mg, about 10 mg to about 25 mg, about 10 mg to about 20 mg, about 10 mg to about 15 mg. In exemplary aspects, the daily dose of astaxanthin is about 20 mg to about 30 mg, e.g., about 21 mg, about 22 mg, about 23 mg, about 24 mg, about 25, mg, about 26 mg, about 27 mg, about 28 mg, about 29 mg, or about 30 mg. Alternatively, the daily dose is about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 75 mg to about 80 mg, about 80 mg to about 115 mg, about 85 mg to about 115 mg, about 90 mg to about 115 mg, about 95 mg to about 115 mg, about 100 mg to about 115 mg, about 105 mg to about 115 mg, about 110 mg to about 115 mg. In exemplary aspects, the daily dose is about is about 90 mg to about 100 mg, e.g., about 91 mg, about 92 mg, about 93 mg, about 94 mg, about 95 mg, about 96 mg, about 97 mg, about 98 mg, about 99 mg, about 100 mg.

In exemplary aspects, the dose of astaxanthin administered to the subject is a twice daily dose. In exemplary aspects, the twice daily dose is about 6 mg to about 20 mg, e.g., about 8 mg to about 20 mg, about 10 mg to about 20 mg, about 12 mg to about 20 mg, about 14 mg to about 20 mg, about 16 mg to about 20 mg, about 18 mg to about 20 mg, about 6 mg to about 18 mg, about 6 mg to about 16 mg, about 6 mg to about 14 mg, about 6 mg to about 12 mg, about 6 mg to about 10 mg, or about 6 mg to about 8 mg. In exemplary aspects, the twice daily dose is about 10 mg to about 15 mg, e.g., about 11 mg, about 12 mg, about 13 mg, about 14 mg. Alternatively, the twice daily dose is about 30 mg to about 60 mg, e.g., about 35 mg to about 60 mg, about 40 mg to about 60 mg, about 45 mg to about 60 mg, about 50 mg to about 60 mg, about 55 mg to about 60 mg, about 30 mg to about 55 mg, about 30 mg to about 50 mg, about 30 mg to about 45 mg, about 30 mg to about 40 mg, about 30 mg to about 35 mg. In exemplary aspects, the twice daily dose is about 40 mg to about 50 mg, e.g., about 41 mg, about 42 mg, about 43 mg, about 44 mg, about 45 mg, about 46 mg, about 47 mg, about 48 mg, about 49 mg, about 50 mg.

In alternative or additional aspects, the amount of astaxanthin administered results in a level of astaxanthin in the bloodstream in the range of 0.1 to 1 μg/mL in plasma or serum. In exemplary aspects, the astaxanthin is administered to achieve a plasma or serum level of about 0.1 μg astaxanthin/mL plasma or serum to about 0.9 μg astaxanthin/mL plasma or serum, about 0.1 μg astaxanthin/mL plasma or serum to about 0.8 μg astaxanthin/mL plasma or serum, about 0.1 μg astaxanthin/mL plasma or serum to about 0.7 μg astaxanthin/mL plasma or serum, about 0.1 μg astaxanthin/mL plasma or serum to about 0.6 μg astaxanthin/mL plasma or serum, about 0.1 μg astaxanthin/mL plasma or serum to about 0.5 μg astaxanthin/mL plasma or serum, about 0.1 μg astaxanthin/mL plasma or serum to about 0.4 μg astaxanthin/mL plasma or serum, about 0.1 μg astaxanthin/mL plasma or serum to about 0.3 μg astaxanthin/mL plasma or serum, about 0.1 μg astaxanthin/mL plasma or serum to about 0.2 μg astaxanthin/mL plasma or serum, about 0.2 μg astaxanthin/mL plasma or serum to about 1.0 μg astaxanthin/mL plasma or serum, about 0.3 μg astaxanthin/mL plasma or serum to about 1.0 μg astaxanthin/mL plasma or serum, about 0.4 μg astaxanthin/mL plasma or serum to about 1.0 μg astaxanthin/mL plasma or serum, about 0.5 μg astaxanthin/mL plasma or serum to about 1.0 μg astaxanthin/mL plasma or serum, about 0.6 μg astaxanthin/mL plasma or serum to about 1.0 μg astaxanthin/mL plasma or serum, about 0.7 μg astaxanthin/mL plasma or serum to about 1.0 μg astaxanthin/mL plasma or serum, about 0.8 μg astaxanthin/mL plasma or serum to about 1.0 μg astaxanthin/mL plasma or serum, about 0.9 μg astaxanthin/mL plasma or serum to about 1.0 μg astaxanthin/mL plasma or serum.

In some aspects, the amount of astaxanthin administered results in a level of astaxanthin in the bloodstream in the range of 1 to 10 μg/mL in plasma or serum. In exemplary aspects, the astaxanthin is administered to achieve a plasma or serum level of about 1 μg astaxanthin/mL plasma or serum to about 9 μg astaxanthin/mL plasma or serum, about 1 μg astaxanthin/mL plasma or serum to about 8 μg astaxanthin/mL plasma or serum, about 1 μg astaxanthin/mL plasma or serum to about 7 μg astaxanthin/mL plasma or serum, about 1 μg astaxanthin/mL plasma or serum to about 6 μg astaxanthin/mL plasma or serum, about 1 μg astaxanthin/mL plasma or serum to about 5 μg astaxanthin/mL plasma or serum, about 1 μg astaxanthin/mL plasma or serum to about 4 μg astaxanthin/mL plasma or serum, about 1 μg astaxanthin/mL plasma or serum to about 3 μg astaxanthin/mL plasma or serum, about 1 μg astaxanthin/mL plasma or serum to about 2 μg astaxanthin/mL plasma or serum, about 2 μg astaxanthin/mL plasma or serum to about 10 μg astaxanthin/mL plasma or serum, about 3 μg astaxanthin/mL plasma or serum to about 10 μg astaxanthin/mL plasma or serum, about 4 μg astaxanthin/mL plasma or serum to about 10 μg astaxanthin/mL plasma or serum, about 5 μg astaxanthin/mL plasma or serum to about 10 μg astaxanthin/mL plasma or serum, about 6 μg astaxanthin/mL plasma or serum to about 10 astaxanthin/mL plasma or serum, about 7 μg astaxanthin/mL plasma or serum to about 10 μg astaxanthin/mL plasma or serum, about 8 μg astaxanthin/mL plasma or serum to about 10 μg astaxanthin/mL plasma or serum, about 9 μg astaxanthin/mL plasma or serum to about 10 μg astaxanthin/mL plasma or serum.

Inflammatory Health and Biomarkers Thereof

Further provided are methods of supporting inflammatory health in a subject at risk for or suffering from CVD. In exemplary embodiments, the methods prevent or treat CVD by reducing CRP levels. In exemplary embodiments, the method comprises administering astaxanthin to the subject. In some embodiments, the subject is one who also receives a standard of care for CVD or CVD risk management.

The present disclosure additionally provides methods of modifying levels of inflammatory health biomarkers or cardiovascular health biomarkers in a subject. In exemplary embodiments, the method (a) reduces levels of one or more of the following: tumor necrosis factor-α (TNF-α), Interleukin-1beta (IL-1β), Interleukin-6 (IL-6), Interferon (IFN-γ), oxidized LDL, total cholesterol, LDL cholesterol, VLDL cholesterol, triglycerides, HemoglobinA1c (HbA1c), alanine aminotransferase (ALT), Aspartate Aminotransferase (AST), body weight, or blood pressure; and/or (b) increases levels of Forkhead box O3 (FOXO3) activation or HDL cholesterol. In some aspects, the method reduces levels of non-HDL cholesterol (total cholesterol minus HDL cholesterol). In exemplary aspects, the method comprises administering astaxanthin. In exemplary embodiments of such methods of modifying levels of biomarkers in a subject, the subject also receives standard of care for CVD or CVD risk management.

As used herein, the phrase “reduces levels” or “reducing levels” or similar phrase may refer to any decrease or reduction in level for the recited biomarker. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease. As used herein, the phrase “increases levels” or “increasing levels” or similar phrase may refer to any increase in level for the recited biomarker. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase.

Suitable methods of determining levels of protein biomarkers (e.g., TNF-α, IL-113, IL-6, INF-γ, ALT, HbA1c, and AST) are known in the art and include immunoassays (e.g., Western blotting, an enzyme-linked immunosorbent assay (ELISA), a radioimmunoassay (RIA), and immunohistochemical assay) or bead-based multiplex assays, e.g., those described in Djoba Siawaya J F, Roberts T, Babb C, Black G, Golakai H J, Stanley K, et al. (2008) An Evaluation of Commercial Fluorescent Bead-Based Luminex Cytokine Assays. PLoS ONE 3(7): e2535. Proteomic analysis which is the systematic identification and quantification of proteins of a particular biological system are known. Mass spectrometry is typically the technique used for this purposes. Methods of measuring biomarker levels are described herein in Examples.

Methods of measuring oxidized low-density lipoprotein (LDL), total cholesterol, LDL cholesterol, very low-density lipoprotein (VLDL) cholesterol, triglycerides, FOXO3 activation or high-density lipoprotein (HDL) cholesterol are known in the art. See, e.g., Park et al., Oncotarget 7(27): 42110-42125 (2016); Chae et al. Sci Rep 8:284 (2018); Lei, et al., Front. Physiol. 7, 270 (2016); and Ghosh et al., Indian J Clin Biochem 21(1): 181-184 (2006).

The measurement of body weight may be accomplished using a scale. In some aspects, the measurement of blood pressure is accomplished by using a blood pressure monitor.

In alternative or additional embodiments, the method of modifying levels of inflammatory health biomarkers in a subject comprises administering astaxanthin at an amount of about 24 mg/day or about 12 mg twice a day.

In exemplary aspects, the dose of astaxanthin is about 10 mg to about 40 mg, e.g., about 15 mg to about 40 mg, about 20 mg to about 40 mg, about 25 mg to about 40 mg, about 30 mg to about 40 mg, about 35 mg to about 40 mg, about 10 mg to about 35 mg, about 10 mg to about 30 mg, about 10 mg to about 25 mg, about 10 mg to about 20 mg, about 10 mg to about 15 mg. In exemplary aspects, the daily dose of astaxanthin is about 20 mg to about 30 mg, e.g., about 21 mg, about 22 mg, about 23 mg, about 24 mg, about 25, mg, about 26 mg, about 27 mg, about 28 mg, about 29 mg, or about 30 mg. Alternatively, the daily dose is about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 75 mg to about 80 mg, about 80 mg to about 115 mg, about 85 mg to about 115 mg, about 90 mg to about 115 mg, about 95 mg to about 115 mg, about 100 mg to about 115 mg, about 105 mg to about 115 mg, about 110 mg to about 115 mg. In exemplary aspects, the daily dose is about is about 90 mg to about 100 mg, e.g., about 91 mg, about 92 mg, about 93 mg, about 94 mg, about 95 mg, about 96 mg, about 97 mg, about 98 mg, about 99 mg, about 100 mg.

In exemplary aspects, the dose of astaxanthin administered to the subject is a twice daily dose. In exemplary aspects, the twice daily dose is about 6 mg to about 20 mg, e.g., about 8 mg to about 20 mg, about 10 mg to about 20 mg, about 12 mg to about 20 mg, about 14 mg to about 20 mg, about 16 mg to about 20 mg, about 18 mg to about 20 mg, about 6 mg to about 18 mg, about 6 mg to about 16 mg, about 6 mg to about 14 mg, about 6 mg to about 12 mg, about 6 mg to about 10 mg, or about 6 mg to about 8 mg. In exemplary aspects, the twice daily dose is about 10 mg to about 15 mg, e.g., about 11 mg, about 12 mg, about 13 mg, about 14 mg. Alternatively, the twice daily dose is about 30 mg to about 60 mg, e.g., about 35 mg to about 60 mg, about 40 mg to about 60 mg, about 45 mg to about 60 mg, about 50 mg to about 60 mg, about 55 mg to about 60 mg, about 30 mg to about 55 mg, about 30 mg to about 50 mg, about 30 mg to about 45 mg, about 30 mg to about 40 mg, about 30 mg to about 35 mg. In exemplary aspects, the twice daily dose is about 40 mg to about 50 mg, e.g., about 41 mg, about 42 mg, about 43 mg, about 44 mg, about 45 mg, about 46 mg, about 47 mg, about 48 mg, about 49 mg, about 50 mg.

In alternative or additional aspects, the amount of astaxanthin administered results in a level of astaxanthin in the bloodstream in the range of 0.1 to 1 μg/mL in plasma or serum. In exemplary aspects, the astaxanthin is administered to achieve a plasma or serum level of about 0.1 μg astaxanthin/mL plasma or serum to about 0.9 μg astaxanthin/mL plasma or serum, about 0.1 μg astaxanthin/mL plasma or serum to about 0.8 μg astaxanthin/mL plasma or serum, about 0.1 μg astaxanthin/mL plasma or serum to about 0.7 μg astaxanthin/mL plasma or serum, about 0.1 μg astaxanthin/mL plasma or serum to about 0.6 μg astaxanthin/mL plasma or serum, about 0.1 μg astaxanthin/mL plasma or serum to about 0.5 μg astaxanthin/mL plasma or serum, about 0.1 μg astaxanthin/mL plasma or serum to about 0.4 μg astaxanthin/mL plasma or serum, about 0.1 μg astaxanthin/mL plasma or serum to about 0.3 μg astaxanthin/mL plasma or serum, about 0.1 μg astaxanthin/mL plasma or serum to about 0.2 μg astaxanthin/mL plasma or serum, about 0.2 μg astaxanthin/mL plasma or serum to about 1.0 μg astaxanthin/mL plasma or serum, about 0.3 μg astaxanthin/mL plasma or serum to about 1.0 μg astaxanthin/mL plasma or serum, about 0.4 μg astaxanthin/mL plasma or serum to about 1.0 μg astaxanthin/mL plasma or serum, about 0.5 μg astaxanthin/mL plasma or serum to about 1.0 μg astaxanthin/mL plasma or serum, about 0.6 μg astaxanthin/mL plasma or serum to about 1.0 μg astaxanthin/mL plasma or serum, about 0.7 μg astaxanthin/mL plasma or serum to about 1.0 μg astaxanthin/mL plasma or serum, about 0.8 μg astaxanthin/mL plasma or serum to about 1.0 μg astaxanthin/mL plasma or serum, about 0.9 μg astaxanthin/mL plasma or serum to about 1.0 μg astaxanthin/mL plasma or serum.

In some aspects, the amount of astaxanthin administered results in a level of astaxanthin in the bloodstream in the range of 1 to 10 μg/mL in plasma or serum. In exemplary aspects, the astaxanthin is administered to achieve a plasma or serum level of about 1 μg astaxanthin/mL plasma or serum to about 9 μg astaxanthin/mL plasma or serum, about 1 μg astaxanthin/mL plasma or serum to about 8 μg astaxanthin/mL plasma or serum, about 1 μg astaxanthin/mL plasma or serum to about 7 μg astaxanthin/mL plasma or serum, about 1 μg astaxanthin/mL plasma or serum to about 6 μg astaxanthin/mL plasma or serum, about 1 μg astaxanthin/mL plasma or serum to about 5 μg astaxanthin/mL plasma or serum, about 1 μg astaxanthin/mL plasma or serum to about 4 μg astaxanthin/mL plasma or serum, about 1 μg astaxanthin/mL plasma or serum to about 3 μg astaxanthin/mL plasma or serum, about 1 μg astaxanthin/mL plasma or serum to about 2 μg astaxanthin/mL plasma or serum, about 2 μg astaxanthin/mL plasma or serum to about 10 μg astaxanthin/mL plasma or serum, about 3 μg astaxanthin/mL plasma or serum to about 10 μg astaxanthin/mL plasma or serum, about 4 μg astaxanthin/mL plasma or serum to about 10 μg astaxanthin/mL plasma or serum, about 5 μg astaxanthin/mL plasma or serum to about 10 μg astaxanthin/mL plasma or serum, about 6 μg astaxanthin/mL plasma or serum to about 10 μg astaxanthin/mL plasma or serum, about 7 μg astaxanthin/mL plasma or serum to about 10 μg astaxanthin/mL plasma or serum, about 8 μg astaxanthin/mL plasma or serum to about 10 μg astaxanthin/mL plasma or serum, about 9 μg astaxanthin/mL plasma or serum to about 10 μg astaxanthin/mL plasma or serum.

In alternative or additional embodiments, the method comprises administering astaxanthin at an amount of about 96 mg/day or about 48 mg twice a day. In exemplary embodiments, the subject is at risk for or suffers from CVD. In additional or alternative embodiments, the method comprises administering astaxanthin at an amount that results in a therapeutic level of astaxanthin in the bloodstream in the range of about 0.1 to about 1 μg/mL in plasma or serum. In additional or alternative embodiments, the method comprises administering astaxanthin at an amount that results in a therapeutic level of astaxanthin in the bloodstream in the range of 1 to 10 μg/mL in plasma or serum. In exemplary embodiments of such methods of modifying biomarker levels, the subject is at risk for or suffers from CVD.

Subjects

In exemplary aspects, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less.

In exemplary aspects, the subject is suffering from a CVD or has a medical history of suffering from CVD, or a sign or symptom thereof. In some aspects, the subject is diagnosed with a CVD. In some instances, the CVD is one of the CVD described herein. In exemplary aspects, the subject has (or had) one of more of the following: myocardial infarction, coronary artery bypass graft (CABG), angioplasty, an intra-coronary artery stent, angina, stroke, transient ischemic attack, atherosclerotic carotid artery disease. In exemplary instances, the subject suffers from Diabetes Mellitus Type II, hypertension, hyperlipidemia, or obesity, or is a smoker. In some instances, the subject suffers from Diabetes Mellitus Type II and is taking an anti-diabetic agent. In some aspects, the subject is also administered one of more of the following cardiac therapeutics: a statin, a hypertension medication, niacin, fibrate, CETP inhibitor, an anti-diabetic agent, a vasoactive agent. In some aspects, the method comprises a combination treatment comprising astaxanthin and another therapeutic agent as described herein.

The following examples are given merely to illustrate the present invention and not in any way to limit its scope.

### EXAMPLES

### Example 1

This example describes the overall study design and plan for the clinical study entitled, Cardiovascular Health: Astaxanthin Supplement Evaluation (CHASE).

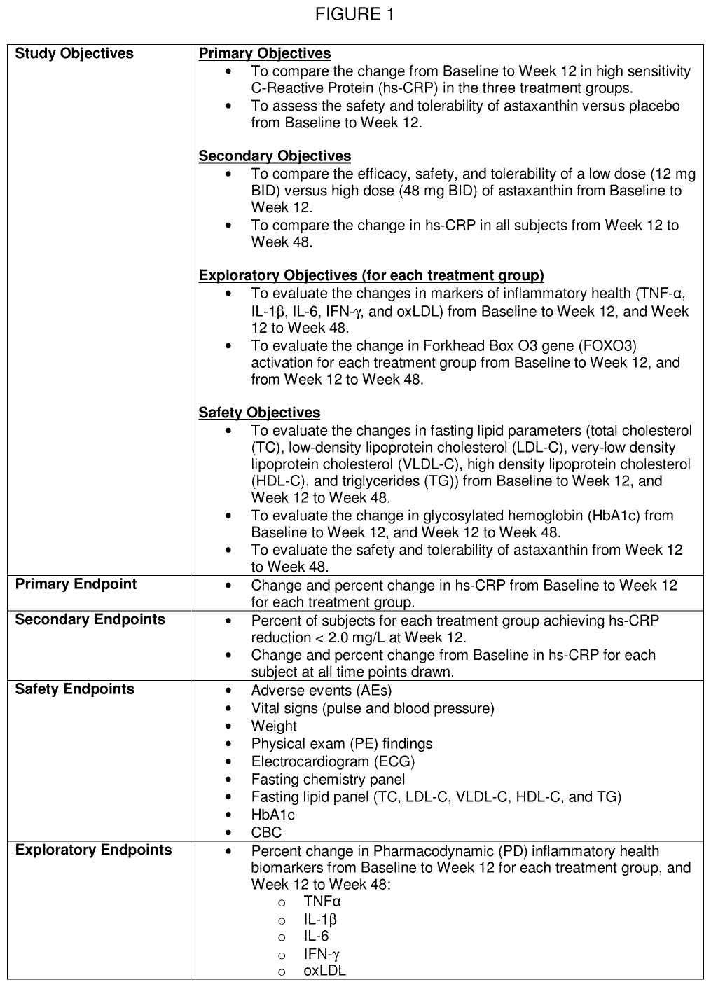

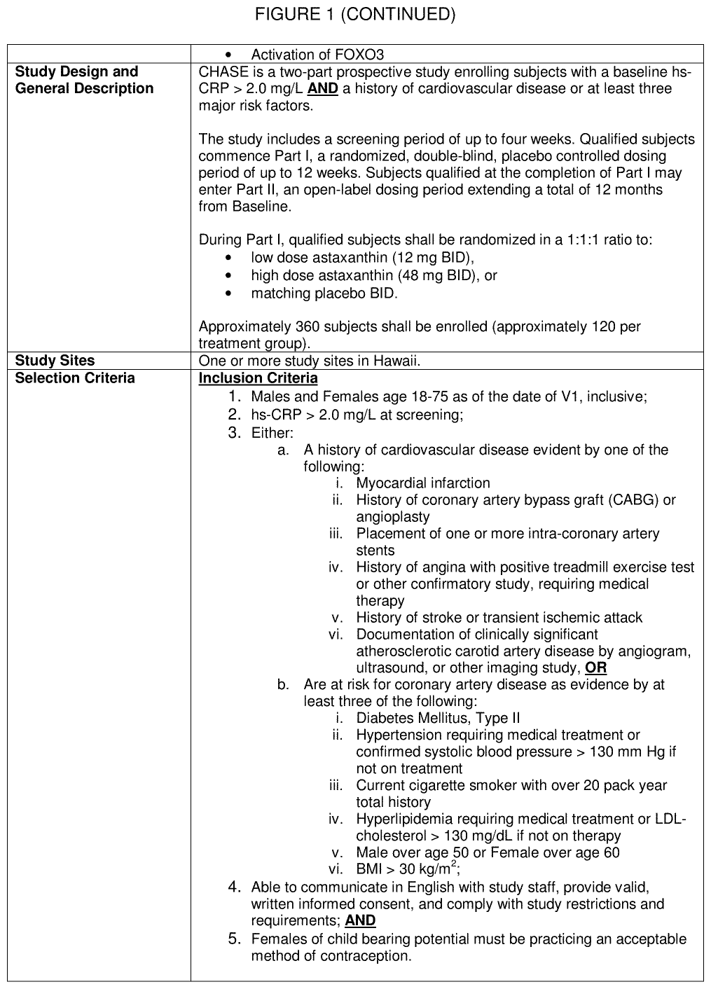

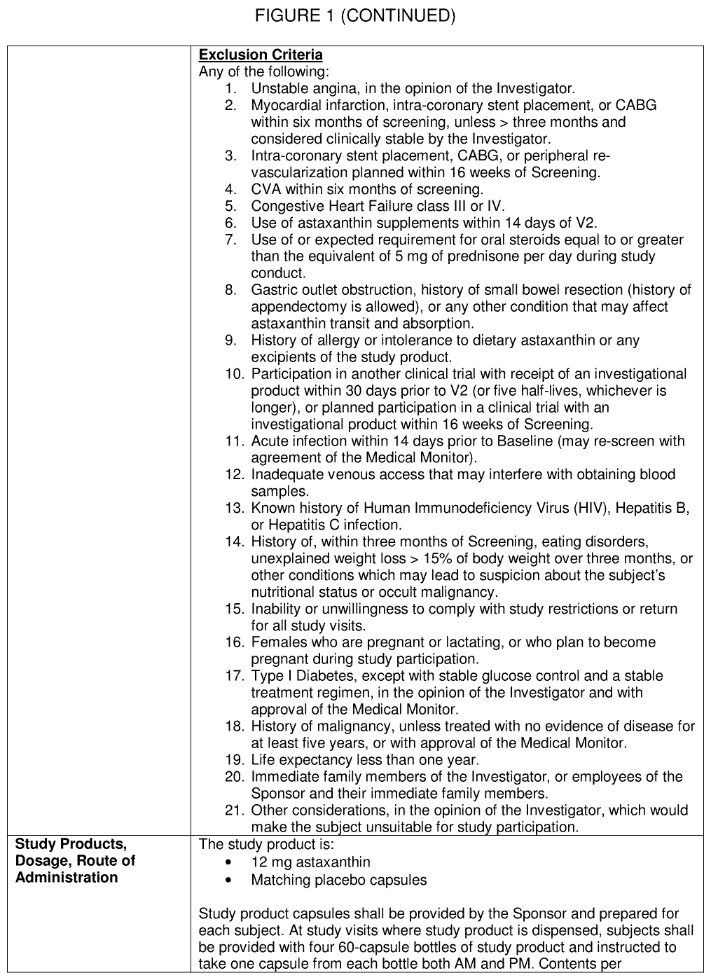

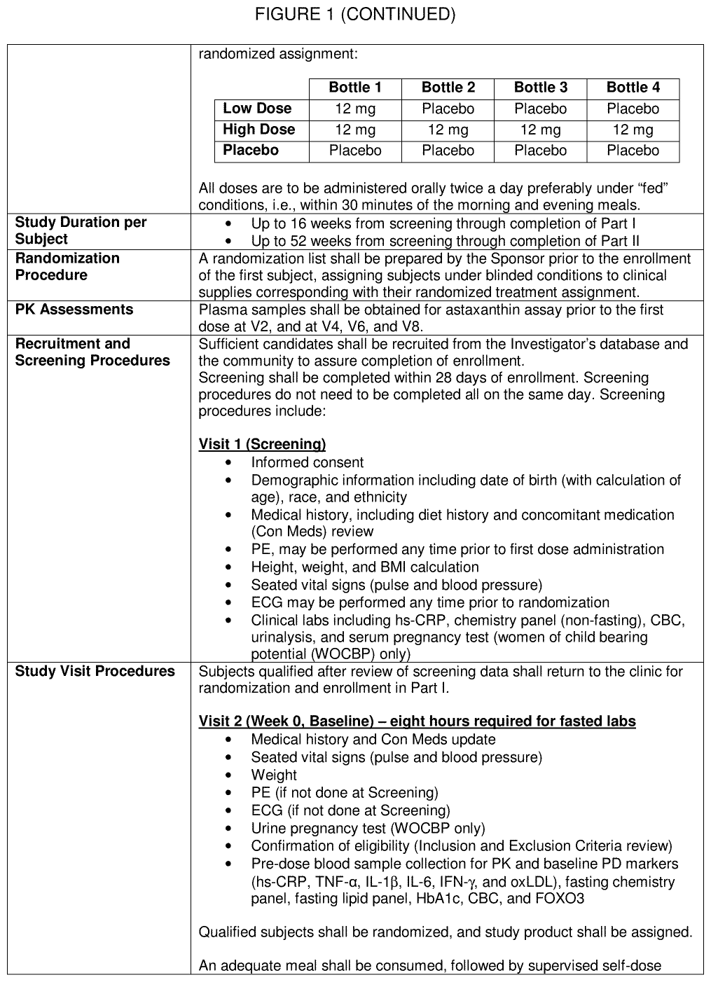

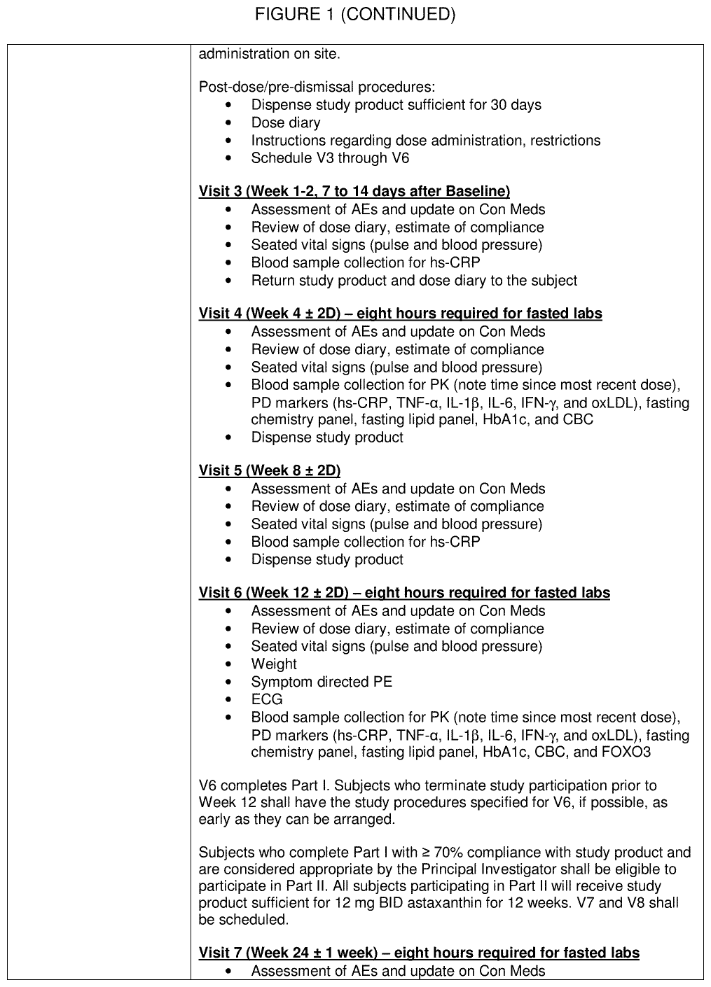

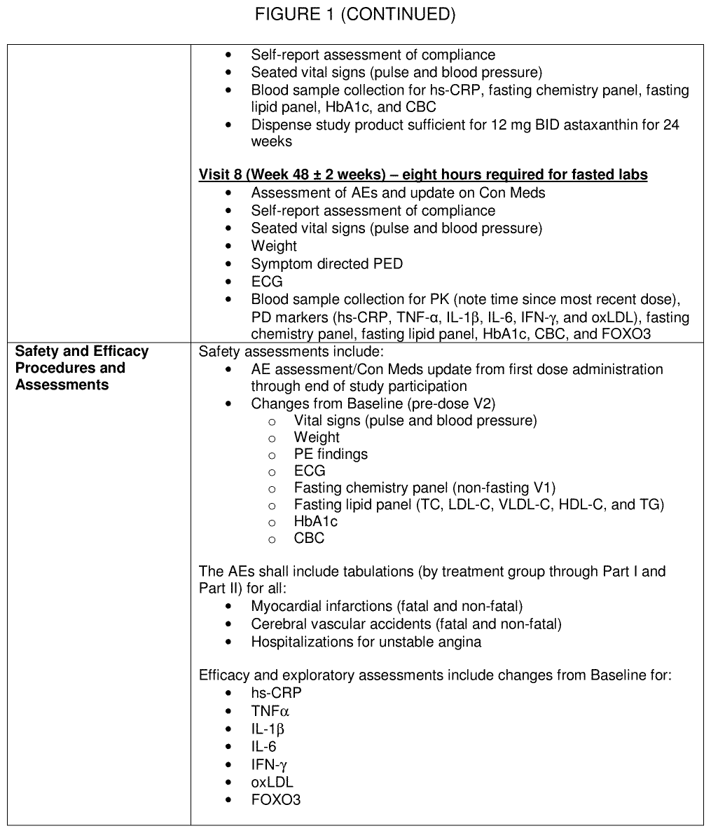

A synopsis of the CHASE study is provided in FIG. 1.

This is a two-part study comprising Part I and Part II. Following screening, Part I consists of a randomized, double-blind, placebo controlled dosing period of up to 12 weeks. The study shall be conducted at one or more study sites in Hawaii. Approximately 360 subjects qualified after completion of screening procedures shall be randomized in a 1:1:1 ratio to one of three treatment groups, per Table 1.

TABLE 1
Treatment Group and Randomized Dose of Study Product

| Treatment Group | Study Product |
|---|---|
| Low Dose | Astaxanthin 12 mg BID |
| High Dose | Astaxanthin 48 mg BID |
| Placebo | Placebo BID |

The Part I 12-week double-blind dosing period commences with V2, including randomization and first dose of study product, and continues with study visits at Week 1-2 (V3), Week 4 (V4), Week 8 (V5), and Week 12 (V6). V6 procedures will conclude Part I.

Study subjects qualified at the completion of Part I may participate in Part II, an open-label dosing period extending through 48 weeks post Baseline. Qualified subjects will be provided with study product to begin Part II, up to 36 additional weeks of open label treatment with 12 mg BID of astaxanthin. Part II includes visits at Week 24 (V7) and Week 48 (V8), which shall conclude participation in this study.

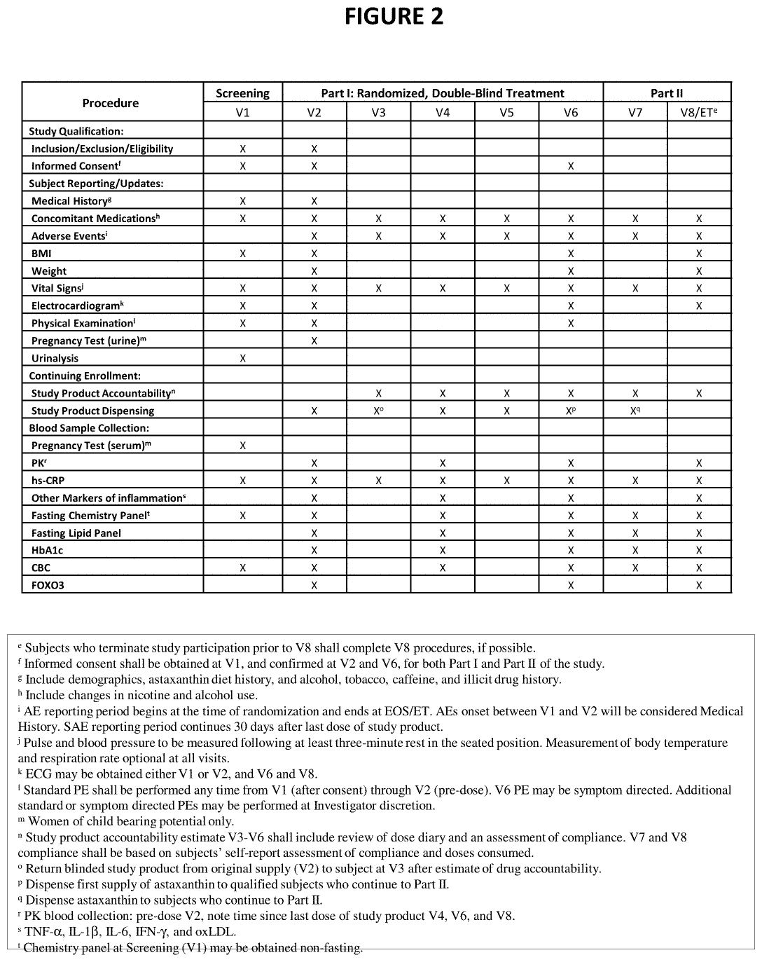

All visits shall be completed on an outpatient basis, without the need for overnight confinement. Details of the schedule and procedures are noted in the Schedule of Activities (FIG. 2).

Randomization and Blinding

Randomization codes shall be generated via a central statistical process and mapped to the subject study number. Study numbers will be matched to study participants sequentially in the order they qualify at V2. The codes will be matched to bottles containing the appropriate study product, and the bottles shall be packaged by study number such that the proper product will be dispensed to each subject to match their randomized treatment group assignment.

The central pharmacy shall maintain the randomization codes and matching subject study numbers. This information shall be available to the Investigator(s) if needed in case of emergency. Any study subjects who have their treatment assignment unblinded during the first 12 weeks of double-blind therapy shall have documentation of the reason for unblinding.

Visit and Procedure Schedule

Visit 1: Screening 28 Days Before Baseline)

Subjects shall be recruited through the Investigator's patient database, advertisements, personal referrals, or other conventional mechanisms. Screening is permitted up to 28 days prior to randomization, which occurs at V2 just before the first dose administration of study product.

Informed Consent

Informal discussion about the study may occur as appropriate in routine clinical contact between the Investigator(s), clinic staff, and clinic patients.

Written informed consent for the study will be obtained by qualified study staff after appropriate discussion and verification of identity from all subjects before all other Protocol-specific procedures are performed. The informed consent form (ICF) will be approved (along with the Protocol and related materials) by an institutional review board (IRB) and will be acceptable to the Investigator and the Sponsor.

The Investigator (or designee) will explain the nature of the study and the study product, including potential risks and benefits and their rights as a research subject. The subjects will be informed that participation is voluntary and that they can withdraw from the study at any time. In accordance with 21 Code of Federal Regulations (CFR) 50, the informed consent process requires adequate discussion and shall be documented by the use of a written ICF approved by the IRB and signed by the subject before Protocol-specific procedures being performed.

The subject will either sign two original ICFs, and be provided with one of the signed ICFs, or sign one ICF, which will be copied. The copy of the signed ICF will be given to the subject and the original will be maintained along with the subject's study records. Informed consent is a process and shall be affirmed through appropriate discussion as subjects continue in the study. Electronic documents/signatures may be utilized with assurance of proper signature.

Screening Procedures

At the screening visit, for subjects who have provided a valid informed consent, the following procedures shall be performed:
Obtain informed consent
Obtain demographic information including date of birth (with calculation of age), race, and ethnicity
Review medical history, concomitant medications (Con Meds) including dietary and herbal supplements, and astaxanthin-related diet history within the past three months
Perform a PE (may be performed at any time after obtaining informed consent and prior to randomization)
Measure height and weight, with calculation of BMI in kg/m2
Measure seated vital signs (pulse and blood pressure)
Single tracing supine 12-lead Electrocardiogram (ECG; may be done any time prior to first dose of study product; may be repeated if necessary for technical reliability)
Clinical lab tests including CRP, chemistry panel, CBC, and urinalysis (fasting is not required for lab samples but the approximate time of last meal prior to the blood sample collection will be documented, if possible)
Serum pregnancy test (women of child bearing potential only)

A screening “lab” number shall be assigned to all subjects who provide informed consent. This number shall primarily be used to identify laboratory specimens.

The screening procedures do not need to be performed in the order listed above except that the informed consent must be completed first. Not all procedures need to be performed the same day or at the same visit. Study staff and the Investigator shall review screening data. Determination of eligibility may include discussion with the Medical Monitor as appropriate. Qualified subjects shall be scheduled to return to the study site for V2 within 28 days of Screening.

A “checklist” of inclusion and exclusion criteria shall be initiated at V1 and updated as data become available.

A screening log shall be maintained of all subjects screened, noting whether they were randomized at V2 or screen failed, and the reason for screen fail if they were not randomized.

Part I

Visit 2: Baseline (Week 0)

All study visits may be completed on an outpatient basis and do not require overnight confinement.

Subjects qualified after completion of screening shall return to the study site for each study visit as instructed by the study staff. V2 should be completed within 28 days of V1.

Baseline labs requiring an overnight fast of at least eight hours (water is permitted ad lib) may be drawn up to two days prior to the date of the Baseline visit or at the Baseline visit, at the discretion of the study staff. It is critical that V2 labs be drawn prior to the subject's first dose. Pre-dose procedures shall be completed as outlined below.

Pre-Dose (Order of Procedures May Vary):
Update medical history and Con Meds
Seated vital signs (pulse and blood pressure)
Weight measurement
Physical examination (if not completed previously during screening)
12-lead ECG (if not completed previously during screening)
Urine pregnancy test (women of child bearing potential only)
Pre-dose blood sample collection for PK and baseline PD markers (CRP, TNF-α, IL-1β, IL-6, IFN-γ, and oxLDL), fasting chemistry panel, fasting lipid panel, HbA1c, CBC, and FOXO3 activity (all baseline V2 labs may be drawn up to two days prior to the office visit if more convenient)
Review of inclusion and exclusion criteria, with completion of I/E checklist started at V1, with authorized signature to document eligibility for randomization
Randomization and study number assignment, with selection of study product matching the subject's randomized treatment group assignment (the subject's study number will match the number on the subject's study product package, and this study number will link the subject to the randomized treatment group)
Consumption of an adequate meal or snack, e.g. a meal containing at least 10 grams of fat and at least 200 calories (meals are not required to be standardized)
   Snack products will be provided, although subjects may bring a meal or snack of their preference if they choose to do so
   The requirement for a meal or snack on-site may be waived if the subject has consumed an adequate meal within one hour prior to dose

First Dose Administration:
The staff shall instruct the subjects regarding proper dose administration and dose diary entries
Following meal completion, the first dose shall be self-administered with approximately 240 mL of water (this will generally occur under the supervision of study staff)

Post-Dose:
Subjects will receive study product sufficient for 30 days, a dose diary, and instructions regarding dose administration, dose diary entries, and restrictions
As much as possible, schedule V3 through V6 and provide the subject with a study calendar prior to dismissal from the study site

Visit 3 (Week 1-2)

V3 may occur within seven to fourteen calendar days (inclusive) after V2. Subjects shall return to the study site with their supply of study product and dose diary. Fasting is not required; however, labs may be drawn up to two days prior to the office visit if more convenient.

Procedures:
Assessment of AEs
Update Con Meds
Review dose diary with estimate of compliance
Seated vital signs (pulse and blood pressure)
Blood sample collection for CRP
Return study product and dose diary to the subject, with reminder regarding restrictions and future study visits

Visit 4 (Week 4±2 Days)

Subjects shall return to the study site with their supply of study product and dose diary. Fasting (eight hours) is required (water allowed) for fasted labs. If more convenient, fasting lab samples may be drawn up to two days prior to V4 or at V4, at the discretion of the study staff. If subjects are unable to comply with fasting labs by the time of their V4, they should be obtained within two days after V4.

Procedures:
Assessment of AEs
Update Con Meds
Review dose diary with estimate of compliance
Seated vital signs (pulse and blood pressure)
Blood sample collection for PK (note time since most recent dose), PD markers (CRP, TNF-α, IL-1β, IL-6, IFN-γ, and oxLDL), fasting chemistry panel, fasting lipid panel, HbA1c, and CBC
Dispense a new supply of study product and a new dose diary, with reminders regarding restrictions and future study visits

Visit 5 (Week 8±2 Days)

Subjects shall return to the study site with their supply of study product and dose diary. Fasting is not required; however, labs may be drawn up to two days prior to the office visit if more convenient.

Procedures:
Assessment of AEs
Update Con Meds
Review dose diary with estimate of compliance
Seated vital signs (pulse and blood pressure)
Blood sample collection for CRP
Dispense a new supply of study product and a new dose diary, with reminders regarding restrictions and future study visits

Visit 6 (Week 12±2 Days): End of Part I

Subjects shall return to the study side with their supply of study product and dose diary. V6 labs requiring an overnight fast of at least eight hours (water is permitted ad lib) may be drawn up to two days prior to V6 or at V6, at the discretion of the study staff. If subjects are unable to comply with fasting labs by the time of their V6, they should be obtained within two days after V6. However, it is critical that V6 labs be drawn prior to the subject's first Part II dose.

Procedures:
Assessment of AEs
Update Con Meds
Review dose diary with estimate of compliance
Seated vital signs (pulse and blood pressure)
Weight measurement
ECG
Symptom directed PE
Blood sample collection for PK (note time since most recent dose), PD markers (CRP, TNF-α, IL-1β, IL-6, IFN-γ, and oxLDL), fasting chemistry panel, fasting lipid panel, HbA1c, CBC, and FOXO3 activity

Subjects who terminate study participation prior to Week 12 shall have the study procedures specified for V6, if possible, as early as they can be arranged.

Part II

Following completion of V6 procedures, study subjects may continue with participation in Part II. Requirements for continuation include:
At least 70% compliance with study product administration through Week 12; AND
Approval of the Principal Investigator, i.e., subjects continue to meet the original inclusion/exclusion criteria and are considered likely to benefit from managing inflammatory health.

Subjects may withdraw rather than participating in Part II. Participation in Part II indicates the subject's expression of interest in continuation and agreement to comply with study requirements and restrictions.

Open-label commercially available ZanthoSyn® shall be dispensed as three bottles of 60 capsules, with instruction to take one capsule twice a day with meals. Study subjects who prefer to take more than one capsule twice a day may obtain additional ZanthoSyn® from commercial sources, at their own expense. Subjects do not need to maintain a formal dose diary, but shall be asked to report the doses consumed and estimate their compliance. Subjects shall be reminded of study restrictions and schedule their future study visits (V7 and V8).

Visit 7 (Week 24±1 Week)

Subjects shall return to the study site. Fasting (eight hours) is required (water allowed) for fasted labs. If more convenient, fasting lab samples may be drawn up to two days prior to V7 or at V7, at the discretion of the study staff. If subjects are unable to comply with fasting labs by the time of their V7, they should be obtained within two days after V7. Subjects do not need to bring their supply of ZanthoSyn® or empty bottles.

Procedures:
Assessment of AEs, with attention to those considered probably, or definitely related to study product
Update Con Meds if changes (including dose adjustments) affect study endpoints in the opinion of the Investigator
Self-report assessment of compliance and doses consumed
Seated vital signs (pulse and blood pressure)
Blood sample collection for CRP, fasting chemistry panel, fasting lipid panel, HbA1c, and CBC
Reminder regarding restrictions and the final study visit

Subjects shall be provided with six bottles of 60-count astaxanthin (e.g., ZanthoSyn®) with instruction to take one capsule twice a day with meals. Subjects do not need to maintain a formal dose diary, but shall be asked to report the doses consumed and estimate their compliance. Subjects shall be reminded of study restrictions and their future study visit.

Visit 8 (Week 48, ±2 Weeks)/Early Termination: End of Study

Subjects shall return to the study site for their end of study visit.

Fasting (eight hours) is required (water allowed) for fasted labs. If more convenient, fasting lab samples may be drawn up to two days prior to V8 or at V8, at the discretion of the study staff. If subjects are unable to comply with fasting labs by the time of their V8, they should be obtained within two days after V8 and before their supply of astaxanthin has been exhausted.

Procedures:
Assessment of AEs, with attention to those considered probably, or definitely related to study product
Update Con Meds if changes (including dose adjustments) affect study endpoints in the opinion of the Investigator
Self-report assessment of compliance and doses consumed
Seated vital signs (pulse and blood pressure)
Single tracing ECG
Weight measurement
PE
Blood sample collection for PK (note time since most recent dose), PD markers (CRP, TNF-α, IL-1β, IL-6, IFN-γ, and oxLDL), fasting chemistry panel, fasting lipid panel, HbA1c, CBC, and FOXO3 activity

If subjects are withdrawn from the study prematurely, efforts shall be made to complete these assessments as part of the early termination (ET) visit in a timely manner.

### Example 2

This example describes the assessment procedures that are followed in the CHASE clinical study.

Physical Examination (PE)

PE shall be performed by the Investigator or qualified designees and include the following body systems and regions: general appearance; head, eyes, ears, nose, and throat; skin; cardiovascular; respiratory; gastrointestinal, musculoskeletal; neurological; lymphatic. Baseline abnormalities considered to be stable and “not clinically significant” shall be documented but are not exclusionary.

Symptom Directed PE

The symptom directed physical examination at V6 and V8 may be limited to assessments related to adverse events reported since Baseline. If no adverse events are reported, a physical examination is optional. Additional physical examinations may be performed whenever considered clinically appropriate.

Height and Weight: Weight will be measured wearing a gown or light indoor clothing with pockets empty and shoes removed.

Vital Signs

Following vital signs shall be measured in a seated position after resting at least three minutes with the subject's back supported and both feet touching the floor:
Pulse rate (heart beats per minute)
Systolic and diastolic blood pressure:
   Blood pressure shall be measured in millimeters of mercury (mmHg) with an appropriately sized cuff
   Blood pressure measurements shall preferably use the same arm for each measurement, ordinarily the non-dominant arm
   Use of a calibrated automated sphygmomanometer is allowed

Electrocardiograms (ECG)

A 12-lead ECG shall be obtained at V1 or V2, V6, and V8 using standard technique. Subjects should rest supine for at least five minutes. A single tracing will be sufficient at the specified visits, although the ECG may be repeated if there are technical reasons why the tracing is not satisfactory or considered to be unrepresentative of the subject's overall cardiac status, in the opinion of the Investigator.

Urine Drug Screen, Alcohol, and Cotinine Tests

No urine drug screen, alcohol breath tests, or tests for cotinine are required for this study.

Safety Labs

A chemistry panel, CBC, and urinalysis shall include analytes as specified in Table 2. The chemistry panel may be obtained non-fasting at Screening (V1). The urinalysis at Screening may be performed on site via “dipstick” methods with staff entry of data into the study record.

Additional analytes may be obtained as considered clinically appropriate by the Investigator.

TABLE 2
Safety Lab Analytes

| | | Urinalysis |
|---|---|---|
| Chemistry Panel | CBC | (Dipstick) |
| Sodium | Hemoglobin | Appearance |
| Potassium | Hematocrit | Color |
| Chloride | Red Blood Cell count | Specific gravity |
| Bicarbonate | White Blood Cell count | pH |
| Blood Urea Nitrogen | MCV | Glucose |
| Creatinine | MCHC | Protein |
| Glucose | Platelet count | Ketones |
| Calcium | White Blood Cell | Leukocyte Esterase |
| Aspartate Transaminase | differential | Blood |
| Alanine Transaminase | | Bilirubin |
| Bilirubin | | Urobilinogen |
| Alkaline Phosphatase | | |
| Total Protein | | |
| Albumin | | |

Pharmacodynamic (PD), Exploratory, and Safety Labs

Blood shall be obtained at each visit for CRP. The screening specimen shall be processed at the local lab and reported to the study staff for eligibility determination; CRP samples at V7 and V8 may also be processed locally and reported directly to study staff. Samples for CRP from V2 through V6 may be batched, processed, and reported as specified in the Laboratory Manual.

Samples shall be obtained for TNF-α, IL-1β, IL-6, IFN-γ, and oxLDL at V2, V4, V6, and V8 I.

Samples shall be obtained for FOXO3 at V2, V6, and V8.

Blood samples shall be obtained under fasted conditions for a lipid panel at V2, V4, V6, V7, and V8. This panel shall include total cholesterol, LDL-C, VLDL-C, HDL-C, and triglycerides. A minimum eight-hour fast (water allowed) is required for the lipid panel.

A blood sample shall be obtained for glycosylated hemoglobin (HbA1c) at V2, V4, V6, V7, and V8.

Pharmacokinetic (PK) Assessments

Blood samples shall be collected for astaxanthin analysis at V2, V4, V6, and V8. This Protocol does not necessarily require analysis of all samples for all subjects (e.g., placebo recipients), and the timing for the PK analysis is at Sponsor discretion.

The approximate time of the most recent dose prior to PK sampling shall be documented. PK sampling is sparse, sample acquisition in relationship to the most recent dose will vary, and formal PK calculations (e.g., Tₘₐₓ, Cₘₐₓ, T_{1/2}, AUC) are not possible from the collections scheduled. Analysis will consist of a tabular listing and comparison by treatment group with calculation of the mean, median, standard deviation, maximum, and minimum at the nominal visits. Sub-analysis by subject demographic and clinical characteristics (e.g., gender, age, weight, and smoking status) may be performed at the discretion of the Sponsor.

PK data may be correlated with randomization assignment, compliance records, and PD responses.

PK Sample Specimen Collection

Blood Collection

Blood samples will ordinarily be obtained through direct venipuncture.

Estimated Blood Volume

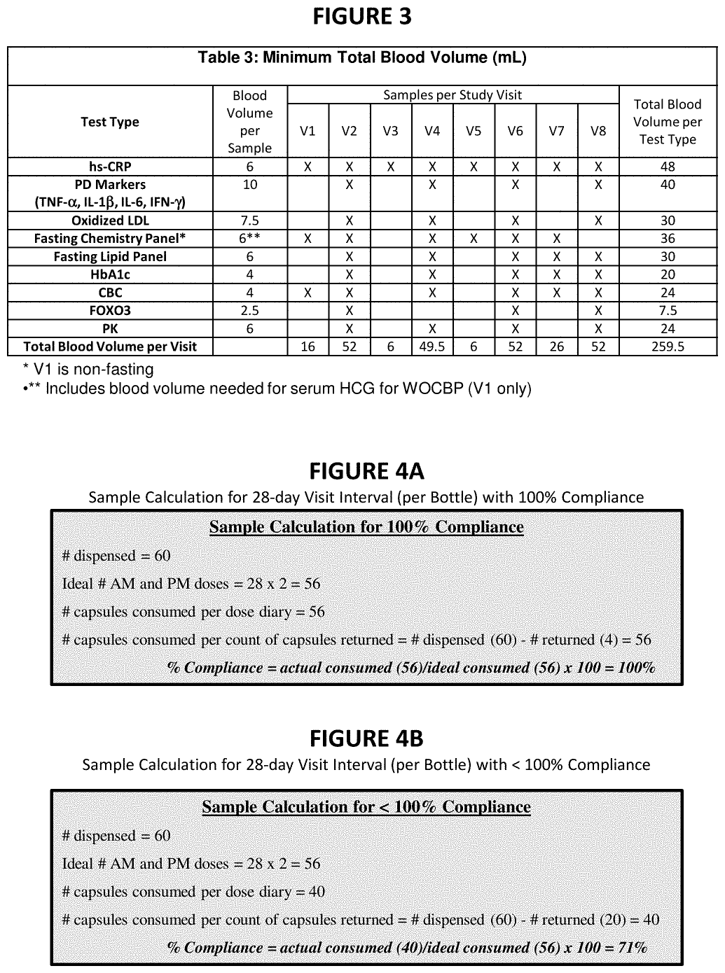

Blood samples are required for PK, PD, and safety lab assessments. Volume estimates are summarized in FIG. 3.

Unscheduled blood samples may be obtained to evaluate adverse events or other standard of care clinical purposes during study conduct if considered appropriate by the Investigator.

PK Sample Processing

PK samples will be collected into a 6.0 mL K₂EDTA tube, gently inverted sufficient times to mix the sample and anticoagulant, and placed in an ice bath for 15 minutes.

The blood samples will be centrifuged at 3500 rpm for ten minutes at room temperature for 10 minutes. Any hemolyzed or clotted samples will be documented in the study data.

All resultant plasma will be split into two approximately equal aliquots and frozen at −60 to −90° C. Samples may be initially placed on dry ice prior to being stored in the appropriate freezer.

PK Sample Specimen Labeling

The samples will be labeled with the study number, subject's lab number, visit number, and the date and time of collection, for correlation with the time of the most recent dose of study drug.

PK Sample Specimen Shipment

PK plasma samples will be retained under frozen conditions. Shipment of specimen batches shall be performed as described in the Laboratory Manual.

Blood Sample Time Windows

The pre-dose PK blood sample at V2 may be obtained up to two days before administration of the first dose. PK blood draws at V4, V6, and V8 are not time sensitive, but the approximate time since the most recent dose of study product should be recorded for each sample.

Fasting lipid profiles and fasting chemistry panels may be obtained within the visit time window on a day separate from the scheduled office visit, within two days before or after the other study procedures. A minimum of eight hours fasted (water allowed ad lib) is required.

PD and safety labs may also be obtained within the time window from the scheduled office visit, although V6 labs must be obtained prior to the commencement of the Part II open-label dosing.

Safety Review

Adverse events and other safety parameters shall be monitored on an ongoing basis by the Investigator(s) and the Medical Monitor.

Data Safety Review Board

A Data Safety Review Board (DSRB) shall be appointed by Sponsor, consisting of the Medical Monitor, the Principal Investigator, and at least three other qualified individuals selected by the Sponsor. The Sponsor shall select the DSRB chairman. The DSRB shall review blinded aggregate safety and PD data at such times as they consider appropriate. Anticipated milestones include:
20% subject completion of V3;
30% subject completion of V4;
50% subject completion of V6; AND
Completion of Part I.

The Sponsor may call for earlier or additional meetings based on their ongoing data review.

Following blinded review, the DSRB may recommend continuation of the study per the Protocol, or request unblinded review of data for one or more study subjects, in which case the Principal Investigator shall ordinarily be excused from the discussion.

Following blinded or (partially) unblinded review, the DSRB may recommend continuation of the study per the Protocol, or if they determine subject safety is at risk, they may recommend:
A temporary hold on dose administration for current subjects, pending further safety review;
A temporary hold on enrollment of new subjects, pending further safety review;
A Protocol amendment;
Study termination; OR
Other action deemed consistent with subject safety.

Alternatively, if an interim review suggests successful demonstration of safety and statistically significant reduction in CRP, additional review may be performed. If the study objectives are met, the Sponsor may suspend or discontinue enrollment prior to randomization of 360 subjects.

### Example 3

This example describes the study product to be used in Parts I and II of the CHASE clinical study.

Part I Study Product

Description of Study Product

The study product active product ingredient (API) during the Part I double-blind dosing period shall contain 12 mg astaxanthin (e.g., from the commercially-available ZanthoSyn® dietary supplement).

The study product placebo capsules shall be matching in size and appearance. Contents and excipients will have no pharmacodynamic activity in relationship to the objectives and endpoints of this study.

Products for this study, both active and placebo, shall be prepared with dark blue size zero capsules specific for this study.

Study Product Packaging, Labeling, Dose, and Randomized Treatment

Active vs. placebo capsules shall be packaged in bottles of 60 capsules each. The capsules in each individual bottle will either be all active or all placebos.

The bottles shall be numbered with a code identifying the contents as either active or placebo, so they may be packaged by study number and dispensed to the subjects according to their randomized treatment assignment during Part I.

Subjects randomized to placebo shall receive four bottles, all containing placebo capsules. Subjects randomized to the low dose of astaxanthin shall receive one bottle with active capsules and three bottles with placebo capsules. Subjects randomized to the high dose astaxanthin shall receive four bottles, all containing active capsules. Bottle contents and treatment group assignment is summarized in Table 4:

TABLE 3
Bottle Contents per Randomized Treatment Group

| Treatment | Bottle Contents (12 mg API per Active Capsule) | | | |
|---|---|---|---|---|
| Group | Bottle 1 | Bottle 2 | Bottle 3 | Bottle 4 |
| Low Dose | Active | Placebo | Placebo | Placebo |
| High Dose | Active | Active | Active | Active |
| Placebo | Placebo | Placebo | Placebo | Placebo |

The four bottles dispensed at V2, V4, and V5 shall be packed together with the proper combination of active vs. placebo contents, and identified by study number and visit number, e.g., if the subject having study number 501 was randomized to the low dose, his/her package would be identified as “Subject Study #501, Visit 2” and contain one bottle with active contents, and three bottles with placebo contents. These packages shall be prepared by unblinded central staff. Blinded study staff simply match the subject study number and visit number with the proper package identifiers.

Subjects will be instructed to take one capsule from each of the four assigned bottles in the morning and the evening. With this schema, subjects randomized to low dose will receive 12 mg BID for a total daily dose of 24 mg, and subjects randomized to high dose will receive 48 mg BID for a total daily dose of 96 mg.

The labels on each bottle shall identify the contents as for investigational use only and include the study number (CDXI-003) and identifying bottle code. Study staff shall add the subject's initials, study number, and date dispensed to each bottle label at the time it is dispensed. Staff may also randomly designate each bottle as “1,” “2,” “3,” or “4” each time a set of four bottles is dispensed, although neither the study staff nor the subject will know the actual capsule contents.

Study Product Storage

Study product should be stored at ambient (15 to 25° C.) temperatures. Subjects should assure their study product is properly secured from use by unauthorized individuals and protected from unnecessary direct exposure to sunlight and/or humidity.

Randomized Treatment Assignment

A master randomization list shall be prepared utilizing standard statistical methods for a sufficient number of subjects to complete enrollment targets. Subjects shall be randomly assigned to low dose, high dose, or placebo treatment groups.

Study Product Unblinding

Subject randomization codes and study product identifier active vs. placebo codes shall remain blinded to study staff other than the central pharmacy, although the randomization code shall be available to the Principal Investigator if necessary. Unblinding is permitted under emergency conditions where the Investigator believes it is in the best interest of the subject to know his/her product assignment. Efforts shall be made to contact the Medical Monitor ahead of such unblinding, if clinically appropriate. The rationale for unblinding must be documented in the subject's study record with note in the trial master file.

Study Product Preparation and Dispensing

The four bottles of study product dispensed at V2, V4, and V5 will be sufficient for 30 days of BID dose administration.

At V2, V4, and V5, a study product-dispensing log shall be maintained noting:
Protocol number (CDXI-003)
Number codes for the bottles dispensed
Subject study number
Subject initials
Visit dispensed (V2, V4, V5)
Date
Initials or name of staff dispensing the study product to the subject

Study Product Administration

Study product shall be self-administered orally with water both morning and evening, twice a day (BID) either with a meal or within approximately 30 minutes of meal completion. From V2 through V6, subjects will be instructed to take one capsule from each of their four bottles in the morning, and one capsule from each of their four bottles in the evening.

Subjects will be advised that it is best to take one capsule from each bottle in a standard sequence and cautioned that they should not have more than one bottle open at a time to prevent confusion if any study drug capsules are spilled.

If an AM dose is missed, it may be taken with the noon meal, and the evening dose may be taken at the usual time. If the AM dose is missed until the time of the evening dose, the evening dose should be taken at the usual time, and a second dose that day may be taken approximately four hours later, with a small meal or snack.

If a PM dose is missed, the subject may take three doses the next day, at the usual time in the AM, at midday with meal or snack, and at the usual time in the PM.

If both doses are missed for one or more days, they are not to be “made up” the following day.

Any missed or late doses should be recorded in the dose diary.

Study Product Accountability

Study staff shall maintain a log of all investigational products received, dispensed, returned, and retained on site during the double-blind study dosing period. Any products inadvertently dropped, damaged, or otherwise not suitable for administration shall be documented.

While the study product is in possession of the study subjects, any spills or other possible confusion regarding the contents of any bottle should be reported to the study staff immediately.

Bottles of study product lost or otherwise unusable may be replaced by study staff after consultation with the central pharmacy to assure the replacement bottles have the appropriate active or placebo capsule contents consistent with the subject's randomized treatment assignment.

Subjects will be advised to return their clinical supplies for estimates of accountability at V3, V4, V5, and V6.

At V3, an informal estimate of compliance may be assessed through inspection of the bottle contents and review of the dose diary. Counting individual capsules retained in each bottle shall not be required.

Drug accountability at V4, V5, and V6 shall be calculated following inspection of the number of capsules in each bottle returned at each visit, in comparison with the subject's dose diary and days (# of doses) elapsed since the study product was dispensed.

FIGS. 4a and 4b provide sample calculations for 100% compliance and less than 100% compliance, respectively.

An estimate of compliance is permissible following count of the capsules returned in just one bottle if the contents of the other three appear to be similar.

At V3, after estimate of drug accountability, subjects shall have the bottles returned to them that were dispensed at V2. At V4, V5, and V6, study staff shall retain the bottles returned.

At V6 the subject's overall compliance since V2 shall be calculated as the average compliance of V4, V5, and V6.

Study Product Disposal

All study product returned at V4, V5, and V6, plus all unused Part I investigational products shall be returned to the Sponsor at the completion of the study and details of the returned products will be documented. The sponsor shall dispose of the product in compliance with regulatory and local standards.

Part II Study Product

At V6, subjects who qualify for Part II of the study will be dispensed three open-label 60-count capsule bottles of astaxanthin (e.g., ZanthoSyn®).

At V7, subjects who plan to continue in the study will be dispensed six open-label 60-count capsule bottles of astaxanthin (e.g., ZanthoSyn®).

No return of unused product or formal study product accountability shall apply during Part II through end of study. Subjects shall self-report the doses consumed and their estimate of compliance.

### Example 4

This example describes the objectives and purpose of CHASE.

| | |
|---|---|
| Primary Objectives | To compare the change from Baseline to Week 12 in |
| | CRP after use of astaxanthin versus placebo |
| | To assess the safety and tolerability of astaxanthin |
| | versus placebo from Baseline to Week 12 |
| Primary Endpoint | Change and percent change in CRP from Baseline to |
| | Week 12 for each treatment group |
| Secondary Objectives | To compare the efficacy, safety, and tolerability of a |
| | low dose (12 mg BID) versus high dose (48 mg BID) of |
| | astaxanthin from Baseline to Week 12 |
| | To compare the change in CRP from Week 12 to |
| | Week 48 in all study subjects |
| Secondary Endpoints | Percent of subjects for each treatment group achieving |
| | CRP reduction <2.0 mg/L at Week 12 |
| | Change and percent change from baseline in CRP for |
| | each subject at all time points drawn |
| Exploratory Objectives for | To evaluate the changes in markers of inflammatory |
| Each Treatment Group | health (TNF-α, IL-1β, IL-6, IFN-γ, and oxLDL) from |
| | Baseline to Week 12, and from Week 12 to Week 48 |
| | To evaluate the change in FOXO3 activation for each |
| | treatment group from Baseline to Week 12, and from |
| | Week 12 to Week 48 |
| Exploratory Endpoints | Percent change in Pharmacodynamic (PD) |
| | inflammatory health biomarkers from Baseline to Week |
| | 12 for each treatment group, and Week 12 to Week 48: |
| | TNF-α |
| | IL-1β |
| | IL-6 |
| | IFN-γ |
| | oxLDL |
| | Activation of FOXO3 |
| Safety Objectives | To evaluate the changes in fasting lipid parameters |
| | (total cholesterol (TC), low-density lipoprotein |
| | cholesterol (LDL-C), very-low density lipoprotein |
| | cholesterol (VLDL-C), high density lipoprotein |
| | cholesterol (HDL-C), and triglycerides (TG)) from |
| | Baseline to Week 12, and Week 12 to Week 48 |
| | To evaluate the change in glycosylated hemoglobin |
| | (HbA1c) from Baseline to Week 12, and Week 12 to |
| | Week 48 |
| | To evaluate the safety and tolerability of astaxanthin |
| | from Week 12 to Week 48 |
| Safety Endpoints | AEs |
| | Vital signs (pulse and blood pressure) |
| | Weight |
| | PE findings |
| | ECG |
| | Fasting chemistry panel (non-fasting V1) |
| | Fasting lipid panel (TC, LDL-C, VLDL-C, HDL-C, |
| | and TG) |
| | HbA1c |
| | CBC |

### Example 5

This example describes the subject inclusion criteria and subject exclusion criteria for CHASE.

This study will enroll approximately 360 males and females who meet all of the inclusion criteria and none of the exclusion criteria listed below.

Subject Inclusion Criteria
1. Males and Females age 18-75, inclusive, as of the date of V1;
2. CRP>2.0 mg/L at screening;
3. Either:
   a. A history of cardiovascular disease evident by one of the following:
      i. Myocardial infarction
      ii. History of coronary artery bypass graft (CABG) or angioplasty
      iii. Placement of one or more intra-coronary artery stents
      iv. History of angina with positive treadmill exercise test or other confirmatory study, requiring medical therapy
      v. History of stroke or transient ischemic attack
      vi. Documentation of clinically significant atherosclerotic carotid artery disease by angiogram, ultrasound, or other imaging study, OR
   b. Are at risk for coronary artery disease as evidence by at least three of the following:
      i. Diabetes Mellitus, Type II
      ii. Hypertension requiring medical treatment or confirmed systolic blood pressure >130 mm Hg if not on treatment
      iii. Current cigarette smoker with over 20 pack year total history
      iv. Hyperlipidemia requiring medical treatment or LDL-cholesterol >130 mg/dL if not on therapy
      v. Male over age 50 or Female over age 60
      vi. BMI>30 kg/m²;
4. Able to communicate in English with study staff, provide valid, written informed consent, and comply with study restrictions and requirements; AND
5. Females of child bearing potential must be practicing an acceptable method of contraception.

Subject Exclusion Criteria
1. Unstable angina, in the opinion of the Investigator
2. Myocardial infarction, intra-coronary stent placement, or CABG within six months of screening, unless>three months and considered clinically stable by the Investigator
3. Intra-coronary stent placement, CABG, or peripheral re-vascularization planned within 16 weeks of Screening
4. CVA within six months of Screening
5. Congestive Heart Failure Class III or IV
6. Use of astaxanthin supplements within 14 days of V2
7. Use of or expected requirement for oral steroids equal to or greater than the equivalent of 5 mg of prednisone per day during study conduct
8. Gastric outlet obstruction, history of small bowel resection (history of appendectomy is allowed), or any other condition that may affect astaxanthin transit and absorption
9. History of allergy or intolerance to dietary astaxanthin or any excipients of the study product
10. Participation in another clinical trial with receipt of an investigational product within 30 days prior to screening (or five half-lives, whichever is longer).
11. Acute infection within 14 days prior to Baseline (may re-screen with agreement of the Medical Monitor)
12. Inadequate venous access that may interfere with obtaining blood samples.
13. Known history of Human Immunodeficiency Virus (HIV), Hepatitis B, or Hepatitis C infection
14. History of, within three months of Screening, eating disorders, unexplained weight loss, or other conditions which may lead to suspicion about the subject's nutritional status or occult malignancy
15. Inability or unwillingness to comply with study restrictions, or return for all study visits
16. Females who are pregnant or lactating, or who plan to become pregnant within the next 12 months
17. Type I Diabetes, except those with stable glucose control and a stable treatment regimen, in the opinion of the Investigator and with approval of the Medical Monitor
18. History of malignancy unless treated with no evidence of disease for at least three years, or with permission of the Medical Monitor
19. Life expectancy less than one year
20. Immediate family members of the Investigator, or employees of the Sponsor and their immediate families
21. Other considerations, in the opinion of the Investigator, which would make the subject unsuitable for study participation

Subject Withdrawal Criteria and Replacement

Subjects may be discontinued from the study prior to completion if any of the following are observed:
Any clinically significant AE
The subject withdraws consent for study participation
The Investigator determines that the subject should not continue
Any other administrative reason

In most circumstances, the Investigator is responsible for determining whether or not an AE warrants discontinuing the subject from the study. The decision should be based on the specifics of the event and the individual subject, and an assessment of the benefits and risks of continued participation in the study. The Medical Monitor may be consulted as needed.

A record will be kept of all subjects who fail to complete the study and the reasons for discontinuation. Subjects who discontinue from the study prematurely will be encouraged to return for early termination assessments. For this study, the End of Study (EOS) assessments will serve as early termination assessments for any subject who withdraws prior to completion.

Alternate Subjects and Subject Replacement

Screening and will continue until enrollment goals are met or an interim analysis demonstrates that the study objectives have been met. Up to 396 subjects may be included if subjects who have completed V1 are qualified when the 360^{th} subject is randomized. Subjects who withdraw or terminate will not be replaced.

### Example 6

This example provides interim results from the CHASE clinical study.

Subjects (n=40) were enrolled in the CHASE clinical study and baseline assessments and screening visits were conducted as described in Example 1-5. A table of the subjects' demographics, diagnoses, and concomitant medications are shown in Tables 6A-6C, respectively.

TABLE 6A

| | | Group X | Group Y | Group Z | |
|---|---|---|---|---|---|
| | | (N = 11) | (N = 10) | (N = 19) | |
| Gender | | | | | |
| Female | n (%) | 5 (45.5%) | 7 (70.0%) | 10 | (52.6%) |
| Male | n (%) | 6 (54.5%) | 3 (30.0%) | 9 | (47.4%) |
| Age (years) | n | 11 | 10 | 19 | |
| | Mean | 60.3 | 60.4 | 58.6 | |
| | SD | 11.21 | 9.45 | 12.42 | |
| | Median | 63.0 | 59.0 | 61.0 | |
| | Min, Max | 36, 75 | 44, 76 | 31, 75 | |
| Age Category | | | | | |
| <60 years | n (%) | 5 (45.5%) | 6 (60.0%) | 9 | (47.4%) |
| =>60 years | n (%) | 6 (54.5%) | 4 (40.0%) | 10 | (52.6%) |
| Ethnicity | | | | | |
| No ethnicity specified | n (%) | 7 (63.6%) | 6 (60.0%) | 6 | (31.6%) |
| Not Hispanic or Latino | n (%) | 4 (36.4%) | 4 (40.0%) | 13 | (68.4%) |
| Race | | | | | |
| Asian | n (%) | 0 (0.0%) | 3 (30.0%) | 6 | (31.6%) |
| Native Hawaiian or Other Pacific | n (%) | 1 (9.1%) | 2 (20.0%) | 4 | (21.1%) |
| Islander | | | | | |
| No race specified | n (%) | 6 (54.5%) | 5 (50.0%) | 5 | (26.3%) |
| White | n (%) | 4 (36.4%) | 0 (0.0%) | 4 | (21.1%) |
| BMI(kg/m²) | n | 11 | 10 | 19 | |
| | Mean | 35.9 | 37.5 | 37.3 | |
| | SD | 5.77 | 9.12 | 7.83 | |
| | Median | 32.9 | 36.3 | 37.6 | |
| | Min, Max | 29.0, 44.6 | 20.8, 54.6 | 28.0, 59.9 | |
| BMI Category | | | | | |
| <30 | n (%) | 2 (18.2%) | 1 (10.0%) | 5 | (26.3%) |
| =>30 | n (%) | 9 (81.8%) | 9 (90.0%) | 14 | (73.7%) |

TABLE 6B

| | | Group X | | Group Y | Group Z | |
|---|---|---|---|---|---|---|
| | | (N = 11) | | (N = 10) | (N = 19) | |
| Coronary Artery Disease | | | | | | |
| No | n (%) | 10 | (90.9%) | 9 (90.0%) | 14 | (73.7%) |
| Yes | n (%) | 1 | (9.1%) | 1 (10.0%) | 5 | (26.3%) |
| Diabetes Mellitus Type II | | | | | | |
| No | n (%) | 5 | (45.5%) | 3 (30.0%) | 7 | (36.8%) |
| Yes | n (%) | 6 | (54.5%) | 7 (70.0%) | 12 | (63.2%) |
| Hypertension | | | | | | |
| No | n (%) | 1 | (9.1%) | 1 (10.0%) | 0 | (0.0%) |
| Yes | n (%) | 10 | (90.9%) | 9 (90.0%) | 19 | (100.0%) |
| Hyperlipidemia | | | | | | |
| No | n (%) | 3 | (27.3%) | 2 (20.0%) | 4 | (21.1%) |
| Yes | n (%) | 8 | (72.7%) | 8 (80.0%) | 15 | (78.9%) |
| Smoker | | | | | | |
| No | n (%) | 9 | (81.8%) | 8 (80.0%) | 13 | (68.4%) |
| Yes | n (%) | 2 | (18.2%) | 2 (20.0%) | 6 | (31.6%) |
| Diabetes Mellitus Type II, | | | | | | |
| hypertension, and hyperlipidemia | | | | | | |
| but not Coronary Artery Disease | | | | | | |
| No | n (%) | 6 | (54.5%) | 6 (60.0%) | 14 | (73.7%) |
| Yes | n (%) | 5 | (45.5%) | 4 (40.0%) | 5 | (26.3%) |
| Don't have Diabetes Mellitus Type | | | | | | |
| II, hypertension, and | | | | | | |
| hyperlipidemia, nor Coronary | | | | | | |
| Artery Disease | | | | | | |
| No | n (%) | 6 | (54.5%) | 5 (50.0%) | 10 | (52.6%) |
| Yes | n (%) | 5 | (45.5%) | 5 (50.0%) | 9 | (47.4%) |
| Baseline hs-CRP | | | | | | |
| <2.0 | n (%) | 1 | (9.1%) | 3 (30.0%) | 5 | (26.3%) |
| =>2.0 | n (%) | 10 | (90.9%) | 7 (70.0%) | 14 | (73.7%) |

TABLE 6C

| | | |
|---|---|---|
| Group X | Group Y | Group Z |
| (N = 11) | (N = 10) | (N = 19) |

Cardiac Therapeutic
Agent

| No | n (%) | 5 | (45.5%) | 4 (40.0%) | 11 | (57.9%) |
|---|---|---|---|---|---|---|
| Yes | n (%) | 6 | (54.5%) | 6 (60.0%) | 8 | (42.1%) |
| Statin | | | | | | |
| No | n (%) | 6 | (54.5%) | 5 (50.0%) | 8 | (42.1%) |
| Yes | n (%) | 5 | (45.5%) | 5 (50.0%) | 11 | (57.9%) |
| Hypertension Medication | | | | | | |
| No | n (%) | 1 | (9.1%) | 2 (20.0%) | 3 | (15.8%) |
| Yes | n (%) | 10 | (90.9%) | 8 (80.0%) | 16 | (84.2%) |
| Anti-diabetic Agent | | | | | | |
| No | n (%) | 5 | (45.5%) | 4 (40.0%) | 8 | (42.1%) |
| Yes | n (%) | 6 | (54.5%) | 6 (60.0%) | 11 | (57.9%) |
| Non-statin Lipid | | | | | | |
| No | n (%) | 10 | (90.9%) | 9 (90.0%) | 18 | (94.7%) |
| Yes | n (%) | 1 | (9.1%) | 1 (10.0%) | 1 | (5.3%) |

Subjects were divided into three treatment groups as outlined in Table 4. Hereinafter, the Low Dose treatment group was referenced as Group X, the Placebo treatment group was referenced as Group Y, and the High Dose treatment group was referenced as Group Z. The activities of Visit 1 (V1), Visit 2 (V2), Visit 3 (V3), Visit 4 (V4), Visit 5 (V5), and Visit 6 (V6) were carried out as essentially described in Example 1.

At each these visits, blood was obtained for measurement of CRP, among other parameters. Table 7A provides a summary of the CRP measurements (mg/L) at each visit and Table 7B provides a summary of the differences of CRP levels between V2 and V6.

TABLE 7A

| | Group X | Group Y | Group Z |
|---|---|---|---|
| | (N = 11) | (N = 10) | (N = 19) |
| V1 | | | |
| N | 11 | 10 | 19 |
| Mean (SD) | 5.43 (3.91) | 4.89 (3.05) | 5.12 (3.39) |
| Median | 3.95 | 3.38 | 4.20 |
| Min, Max | 2.87, 15.96 | 2.15, 11.82 | 2.03, 14.17 |
| V2 | | | |
| N | 11 | 10 | 19 |
| Mean (SD) | 5.52 (4.80) | 6.19 (5.61) | 4.28 (2.32) |
| Median | 3.64 | 4.32 | 4.68 |
| Min, Max | 1.39, 17.86 | 1.23, 17.76 | 1.27, 8.51 |
| V3 | | | |
| N | 11 | 10 | 19 |
| Mean (SD) | 3.72 (2.06) | 6.05 (4.48) | 4.86 (3.09) |
| Median | 3.44 | 4.79 | 4.52 |
| Min, Max | 0.44, 6.72 | 1.62, 13.91 | 1.77, 13.08 |
| V4 | | | |
| N | 11 | 10 | 19 |
| Mean (SD) | 3.98 (4.24) | 5.05 (4.28) | 3.42 (1.84) |
| Median | 2.56 | 2.95 | 3.04 |
| Min, Max | 0.56, 15.83 | 0.86, 12.76 | 0.90, 7.81 |
| V5 | | | |
| N | 11 | 10 | 19 |
| Mean (SD) | 4.90 (4.99) | 5.23 (3.18) | 3.49 (2.06) |
| Median | 3.71 | 4.91 | 3.04 |
| Min, Max | 1.06, 18.24 | 1.66, 9.41 | 0.91, 9.20 |
| V6 | | | |
| N | 11 | 10 | 19 |
| Mean (SD) | 3.45 (3.09) | 5.29 (5.22) | 3.41 (2.24) |
| Median | 2.70 | 3.47 | 2.53 |
| Min, Max | 0.22, 11.04 | 0.76, 17.66 | 0.61, 8.84 |

TABLE 7B

| | Group X | | Group Y | | Group Z | |
|---|---|---|---|---|---|---|
| | (N = 11) | | (N = 10) | | (N = 19) | |
| V2 to V6 Change | | | | | | |
| N | 11 | | 10 | | 19 | |
| Mean (SD) | −2.07 | (4.61) | −0.89 | (4.48) | −0.87 | (2.02) |
| Median | −1.57 | | −0.11 | | −0.65 | |
| Min, Max | −11.1, 6.08 | | −12.7, 4.13 | | −6.07, 2.58 | |
| V2 to V6 % Change | | | | | | |
| N | 11 | | 10 | | 19 | |
| Mean (SD) | −21.3 | (69.63) | −2.56 | (49.50) | −8.48 | (46.86) |

| | | | |
|---|---|---|---|
| Median | −31.8 | −5.16 | −27.9 |
| Min, Max | −89.8, 122.6 | −90.1, 91.57 | −90.9, 81.89 |

Table 7C provides CRP measurements (mg/L) at each visit for subjects with a baseline level greater than 2.0 and Table 7D provides a summary of the differences of CRP levels between V2 and V6.

TABLE 7C

| | Group X | Group Y | Group Z |
|---|---|---|---|
| | (N = 10) | (N = 7) | (N = 14) |
| V1 | | | |
| N | 10 | 7 | 14 |
| Mean (SD) | 5.64 (4.06) | 5.80 (3.27) | 5.65 (3.56) |
| Median | 3.98 | 5.85 | 4.94 |
| Min, Max | 2.87, 15.96 | 2.15, 11.82 | 2.03, 14.17 |
| V2 | | | |
| N | 10 | 7 | 14 |
| Mean (SD) | 5.93 (4.85) | 8.21 (5.58) | 5.25 (1.89) |
| Median | 4.29 | 6.00 | 5.17 |
| Min, Max | 2.16, 17.86 | 3.08, 17.76 | 2.29, 8.51 |
| V3 | | | |
| N | 10 | 7 | 14 |
| Mean (SD) | 3.97 (1.97) | 7.68 (4.42) | 5.62 (3.21) |
| Median | 3.55 | 6.42 | 4.88 |
| Min, Max | 0.44, 6.72 | 2.29, 13.91 | 2.27, 13.08 |
| V4 | | | |
| N | 10 | 7 | 14 |
| Mean (SD) | 4.22 (4.39) | 6.47 (4.40) | 3.94 (1.86) |
| Median | 3.27 | 5.40 | 3.27 |
| Min, Max | 0.56, 15.83 | 1.55, 12.76 | 0.90, 7.81 |
| V5 | | | |
| N | 10 | 7 | 14 |
| Mean (SD) | 5.29 (5.09) | 5.92 (3.30) | 4.09 (2.07) |
| Median | 4.08 | 7.13 | 4.17 |
| Min, Max | 1.06, 18.24 | 1.66, 9.41 | 1.15, 9.20 |
| V6 | | | |
| N | 10 | 7 | 14 |
| Mean (SD) | 3.69 (3.14) | 6.96 (5.46) | 3.88 (2.42) |
| Median | 2.71 | 5.99 | 2.89 |
| Min, Max | 0.22, 11.04 | 1.39, 17.66 | 0.61, 8.84 |

TABLE 7D

| | Group X | Group Y | Group Z |
|---|---|---|---|
| | (N = 10) | (N = 7) | (N = 14) |
| V2 to V6 Change | | | |
| N | 10 | 7 | 14 |
| Mean (SD) | −2.24 (4.83) | −1.25 (5.43) | −1.37 (2.11) |
| Median | −1.60 | −0.10 | −1.54 |
| Min, Max | −11.1, 6.08 | −12.7, 4.13 | −6.07, 2.58 |
| V2 to V6 % Change | | | |
| N | 10 | 7 | 14 |
| Mean (SD) | −20.8 (73.37) | −0.45 (56.87) | −24.8 (35.03) |
| Median | −40.7 | −0.56 | −31.1 |
| Min, Max | −89.8, 122.6 | −90.1, 91.57 | −90.9, 48.77 |

Table 7E provides the change in CRP measurements (mg/L) (V2 to V6) for subjects with Diabetes Mellitus Type II (DM2). Table 7F provides the change in CRP measurements (mg/L) (V2 to V6) for subjects with hyperlipidemia. Table 7G provides the change in CRP measurements (mg/L) (V2 to V6) for subjects with all three of DM2, hypertension and hyperlipidemia but not coronary artery disease (CAD). Table 7H provides the change in CRP measurements (mg/L) (V2 to V6) for subjects with CAD. Table 71 provides the change in CRP measurements (mg/L) (V2 to V6) for subjects not taking any cardiac therapeutic agent. Table 7J provides the change in CRP measurements (mg/L) (V2 to V6) for subjects not taking a hypertension agent.

TABLE 7E

| | Group X | Group Y | Group Z |
|---|---|---|---|
| | (N = 6) | (N = 7) | (N = 12) |
| V2 | | | |
| N | 6 | 7 | 12 |
| Mean (SD) | 7.52 (5.87) | 7.95 (5.90) | 4.78 (2.37) |
| Median | 6.78 | 6.00 | 5.01 |
| Min, Max | 1.39, 17.86 | 1.23, 17.76 | 1.27, 8.51 |
| V6 | | | |
| N | 6 | 7 | 12 |
| Mean (SD) | 2.91 (2.07) | 6.80 (5.63) | 3.77 (2.70) |
| Median | 2.32 | 5.99 | 2.56 |
| Min, Max | 1.02, 6.80 | 1.11, 17.66 | 0.61, 8.84 |
| V2 to V6 Change | | | |
| N | 6 | 7 | 12 |
| Mean (SD) | −4.61 (4.14) | −1.15 (5.44) | −1.01 (2.32) |
| Median | −3.76 | −0.10 | −0.90 |
| Min, Max | −11.1, −0.37 | −12.7, 4.13 | −6.07, 2.58 |
| V2 to V6 % Change | | | |
| N | 6 | 7 | 12 |
| Mean (SD) | −52.9 (20.70) | 2.15 (55.81) | −14.7 (47.59) |
| Median | −55.7 | −0.56 | −29.3 |
| Min, Max | −78.8, −26.6 | −90.1, 91.57 | −90.9, 81.89 |

TABLE 7F

| | Group X | Group Y | Group Z |
|---|---|---|---|
| | (N = 8) | (N = 8) | (N = 15) |
| V2 | | | |
| N | 8 | 8 | 15 |
| Mean (SD) | 6.71 (5.19) | 5.99 (6.32) | 3.78 (2.28) |
| Median | 4.95 | 3.61 | 3.12 |
| Min, Max | 1.39, 17.86 | 1.23, 17.76 | 1.27, 8.51 |
| V6 | | | |
| N | 8 | 8 | 15 |
| Mean (SD) | 3.90 (3.38) | 4.84 (5.79) | 2.54 (1.29) |
| Median | 2.71 | 2.28 | 2.31 |
| Min, Max | 1.02, 11.04 | 0.76, 17.66 | 0.61, 6.14 |
| V2 to V6 Change | | | |
| N | 8 | 8 | 15 |
| Mean (SD) | −2.81 (5.17) | −1.15 (4.91) | −1.24 (1.95) |
| Median | −1.60 | −0.11 | −1.14 |
| Min, Max | −11.1, 6.08 | −12.7, 4.13 | −6.07, 1.29 |
| V2 to V6 % Change | | | |
| N | 8 | 8 | 15 |
| Mean (SD) | −27.5 (63.84) | −4.76 (53.39) | −13.6 (49.58) |
| Median | −40.7 | −5.16 | −30.7 |
| Min, Max | −78.8, 122.6 | −90.1, 91.57 | −90.9, 81.89 |

TABLE 7G

| | Group X | Group Y | Group Z |
|---|---|---|---|
| | (N = 5) | (N = 4) | (N = 5) |
| V2 | | | |
| N | 5 | 4 | 5 |
| Mean (SD) | 7.30 (6.54) | 10.12 (6.86) | 5.26 (2.32) |
| Median | 4.94 | 9.29 | 5.04 |
| Min, Max | 1.39, 17.86 | 4.13, 17.76 | 2.59, 8.51 |
| V6 | | | |
| N | 5 | 4 | 5 |
| Mean (SD) | 2.95 (2.32) | 8.07 (7.05) | 3.38 (1.57) |
| Median | 1.91 | 6.62 | 2.58 |
| Min, Max | 1.02, 6.80 | 1.39, 17.66 | 2.48, 6.14 |
| V2 to V6 Change | | | |
| N | 5 | 4 | 5 |
| Mean (SD) | −4.35 (4.57) | −2.05 (7.33) | −1.87 (1.24) |
| Median | −1.63 | 0.19 | −2.37 |
| Min, Max | −11.1, −0.37 | −12.7, 4.13 | −3.24, −0.11 |
| V2 to V6 % Change | | | |
| N | 5 | 4 | 5 |
| Mean (SD) | −49.7 (21.51) | 3.07 (74.39) | −32.6 (18.99) |
| Median | −49.5 | 5.41 | −30.7 |
| Min, Max | −78.8, −26.6 | −90.1, 91.57 | −50.4, −4.25 |

TABLE 7H

| | Group X | Group Y | Group Z |
|---|---|---|---|
| | (N = 1) | (N = 1) | (N = 5) |
| V2 | | | |
| N | 1 | 1 | 5 |
| Mean (SD) | 8.61 | 1.23 | 3.49 (2.23) |
| Median | 8.61 | 1.23 | 3.12 |
| Min, Max | 8.61, 8.61 | 1.23, 1.23 | 1.27, 6.68 |
| V6 | | | |
| N | 1 | 1 | 5 |
| Mean (SD) | 2.72 | 1.11 | 1.57 (0.72) |
| Median | 2.72 | 1.11 | 1.88 |
| Min, Max | 2.72, 2.72 | 1.11, 1.11 | 0.61, 2.31 |
| V2 to V6 Change | | | |
| N | 1 | 1 | 5 |
| Mean (SD) | −5.89 | −0.12 | −1.91 (2.67) |
| Median | −5.89 | −0.12 | −1.24 |
| Min, Max | −5.89, −5.89 | −1.012, −0.12 | −6.07, 1.04 |
| V2 to V6 % Change | | | |
| N | 1 | 1 | 5 |
| Mean (SD) | −68.4 | −9.76 | −28.8 (65.35) |
| Median | −68.4 | −9.76 | −39.7 |
| Min, Max | −68.48, −68.4 | −9.76, −9.76 | −90.9, 81.89 |

TABLE 7I

| | Group X | Group Y | Group Z |
|---|---|---|---|
| | (N = 5) | (N = 4) | (N = 11) |
| V2 | | | |
| N | 6 | 4 | 11 |
| Mean (SD) | 6.81 (6.86) | 4.97 (2.62) | 4.55 (2.02) |
| Median | 3.64 | 5.07 | 4.68 |
| Min, Max | 1.39, 17.86 | 1.81, 7.95 | 1.81, 8.04 |
| V6 | | | |
| N | 5 | 4 | 11 |
| Mean (SD) | 2.53 (2.56) | 5.29 (2.47) | 3.13 (2.14) |
| Median | 1.91 | 5.30 | 2.53 |
| Min, Max | 0.22, 6.80 | 2.34, 8.23 | 0.61, 8.84 |
| V2 to V6 Change | | | |
| N | 5 | 4 | 11 |
| Mean (SD) | −4.28 (4.63) | 0.32 (1.72) | −1.42 (2.01) |
| Median | −1.94 | 0.50 | −1.24 |
| Min, Max | −11.1, −0.37 | −1.96, 2.23 | −6.07, 1.29 |
| V2 to V6 % Change | | | |
| N | 5 | 4 | 11 |
| Mean (SD) | −56.6 (29.46) | 13.29 (27.50) | −23.7 (41.82) |
| Median | −61.9 | 20.33 | −31.5 |
| Min, Max | −89.8, −25.8 | −24.7, 37.17 | −90.9, 71.27 |

TABLE 7J

| | Group X | Group Y | Group Z |
|---|---|---|---|
| | (N = 1) | (N = 2) | (N = 3) |
| V2 | | | |
| N | 1 | 2 | 3 |
| Mean (SD) | 3.64 | 3.91 (2.96) | 4.52 (1.86) |
| Median | 3.64 | 3.91 | 4.68 |
| Min, Max | 3.64, 3.64 | 1.81, 6.00 | 2.59, 6.30 |
| V6 | | | |
| N | 1 | 2 | 3 |
| Mean (SD) | 2.70 | 5.29 (4.16) | 2.94 (1.20) |
| Median | 2.70 | 5.29 | 2.48 |
| Min, Max | 2.70, 2.70 | 2.34, 8.23 | 2.04, 4.30 |
| V2 to V6 Change | | | |
| N | 1 | 2 | 3 |
| Mean (SD) | −0.94 | 1.38 (1.20) | −1.58 (1.32) |
| Median | −0.94 | 1.38 | −2.00 |
| Min, Max | −0.94, −0.94 | 0.53, 2.23 | −2.64, −0.11 |
| V2 to V6 % Change | | | |
| N | 1 | 2 | 3 |
| Mean (SD) | −25.8 | 33.22 (5.58) | −30.8 (26.09) |
| Median | −25.8 | 33.22 | −31.8 |
| Min, Max | −25.8, −25.8 | 29.28, 37.17 | −56.4, −4.25 |

Table 7K provides the change in CRP measurements (mg/L) (V2 to V6) for subjects not taking statins and Table 7L provides the change in CRP measurements (mg/L) (V2 to V6) for subjects taking an anti-diabetic agent.

TABLE 7K

| | Group X | Group Y | Group Z |
|---|---|---|---|
| V2 | | | |
| N | 6 | 5 | 8 |
| Mean (SD) | 4.04 (2.48) | 4.88 (2.28) | 4.97 (2.51) |
| Median | 3.12 | 4.51 | 4.83 |
| Min, Max | 2.16, 8.61 | 1.81, 7.95 | 1.83, 8.51 |
| V6 | | | |
| N | 6 | 5 | 8 |
| Mean (SD) | 3.86 (3.78) | 5.96 (2.61) | 4.16 (2.57) |
| Median | 2.71 | 5.99 | 3.24 |
| Min, Max | 0.22, 11.04 | 2.34, 8.64 | 1.88, 8.84 |
| V2 to V6 Change | | | |
| N | 6 | 5 | 8 |
| Mean (SD) | −0.18 (4.04) | 1.08 (2.27) | −0.81 (1.42) |
| Median | −0.80 | 0.53 | −0.71 |
| Min, Max | −5.89, 6.08 | −1.96, 4.13 | −2.64, 0.80 |
| V2 to V6 % Change | | | |
| N | 6 | 5 | 8 |
| Mean (SD) | 2.34 (88.58) | 28.95 (42.34) | −14.9 (28.11) |
| Median | −25.6 | 29.28 | −17.6 |
| Min, Max | −89.8, 122.6 | −24.7, 91.57 | −56.4, 19.13 |

TABLE 7L

| | Group X | Group Y | Group Z |
|---|---|---|---|
| | (N = 6) | (N = 6) | (N = 11) |
| V2 | | | |
| N | 6 | 6 | 11 |
| Mean (SD) | 7.52 (5.87) | 6.93 (5.75) | 4.48 (2.24) |
| Median | 6.78 | 5.26 | 4.98 |
| Min, Max | 1.39, 17.86 | 1.23, 17.76 | 1.27, 8.51 |
| V6 | | | |
| N | 6 | 6 | 11 |
| Mean (SD) | 2.91 (2.07) | 7.71 (5.59) | 3.31 (2.28) |
| Median | 2.32 | 7.11 | 2.53 |
| Min, Max | 1.02, 6.80 | 1.11, 17.66 | 0.61, 7.87 |
| V2 to V6 Change | | | |
| N | 6 | 6 | 11 |
| Mean (SD) | −4.61 (4.14) | 0.78 (2.12) | −1.18 (2.36) |
| Median | −3.76 | 0.19 | −1.14 |
| Min, Max | −11.1, −0.37 | −1.96, 4.13 | −6.07, 2.58 |
| V2 to V6 % Change** | | | |
| N | 6 | 6 | 11 |
| Mean (SD) | −52.9 (20.70) | 17.52 (41.85) | −16.9 (49.24) |
| Median | −55.7 | 5.41 | −30.7 |
| Min, Max | −78.8, −26.6 | −24.7, 91.57 | −90.9, 81.89 |

Table 7M provides the changes in lipid levels (mg/dL) (V2 to V6) for subjects.

TABLE 7M

| | | |
|---|---|---|
| Group X | Group Y | Group Z |
| (N = 11) | (N = 10) | (N = 19) |

Cholesterol (mg/dL)

| | | | |
|---|---|---|---|
| V2 | | | |
| N | 11 | 10 | 19 |
| Mean (SD) | 192.0 (65.49) | 180.5 (29.60) | 195.1 (57.82) |
| Median | 180.0 | 176.5 | 190.0 |
| Min, Max | 113., 342.0 | 141.0, 245.0 | 121.0, 316.0 |
| V6 | | | |
| N | 11 | 10 | 18 |
| Mean (SD) | 182.6 (70.21) | 194.4 (35.95) | 169.7 (45.52) |
| Median | 163.0 | 189.5 | 160.5 |
| Min, Max | 117.0, 339.0 | 124.0, 258.0 | 104.0, 284.0 |
| V2 to V6 Change | | | |
| N | 11 | 10 | 18 |
| Mean (SD) | −9.45 (18.08) | 10.90 (18.53) | −18.6 (31.99) |
| Median | −7.00 | 8.00 | −13.5 |
| Min, Max | −39.0, 17.00 | −17.0, 49.00 | −92.0, 32.00 |
| V2 to V6 % Change | | | |
| N | 11 | 10 | 18 |
| Mean (SD) | −5.34 (10.55) | 6.02 (11.45) | −8.52 (14.73) |
| Median | −4.90 | 4.40 | −8.29 |
| Min, Max | −25.0, 6.67 | −12.1, 28.16 | −38.0, 15.86 |

LDL (mg/dL)

| | | | |
|---|---|---|---|
| V2 | | | |
| N | 10 | 9 | 16 |
| Mean (SD) | 111.9 (61.26) | 99.89 (27.99) | 99.13 (46.59) |
| Median | 112.0 | 96.00 | 84.00 |
| Min, Max | 37.00, 220.0 | 63.00, 152.0 | 34.00, 206.0 |
| V6 | | | |
| N | 10 | 10 | 16 |
| Mean (SD) | 109.6 (67.65) | 107.2 (32.10) | 90.31 (37.82) |
| Median | 102.0 | 104.0 | 81.00 |
| Min, Max | 40.00, 236.0 | 65.00, 160.0 | 49.00, 176.0 |
| V2 to V6 Change | | | |
| N | 10 | 9 | 15 |
| Mean (SD) | −2.30 (12.98) | 7.67 (15.93) | −18.9 (24.97) |
| Median | −6.50 | 5.00 | −11.0 |
| Min, Max | −18.0, 17.00 | −8.00, 46.00 | −83.0, 11.00 |
| V2 to V6 % Change | | | |
| N | 10 | 9 | 15 |
| Mean (SD) | −3.46 (13.64) | 7.35 (14.92) | −14.5 (17.99) |
| Median | −7.02 | 4.94 | −12.0 |
| Min, Max | −21.6, 21.62 | −11.0, 41.82 | −50.0, 15.71 |

Triglycerides (mg/dL)

| | | | |
|---|---|---|---|
| V2 | | | |
| N | 11 | 10 | 19 |
| Mean (SD) | 201.9 (122.2) | 172.0 (108.1) | 283.5 (368.2) |
| Median | 177.0 | 151.0 | 175.0 |
| Min, Max | 59.00, 482.0 | 68.00, 444.0 | 75.00, 1677 |
| V6 | | | |
| N | 11 | 10 | 18 |
| Mean (SD) | 177.4 (109.8) | 179.3 (93.83) | 192.2 (155.1) |
| Median | 131.0 | 144.0 | 145.5 |
| Min, Max | 70.00, 420.0 | 72.00, 389.0 | 63.00, 650.0 |
| V2 to V6 Change | | | |
| N | 11 | 10 | 18 |
| Mean (SD) | −24.6 (59.47) | 7.30 (92.47) | −9.89 (109.9) |
| Median | −26.0 | 6.00 | −24.5 |
| Min, Max | −166, 56.00 | −195, 154.0 | −178, 348.0 |
| V2 to V6 % Change | | | |
| N | 11 | 10 | 18 |
| Mean (SD) | −7.10 (27.88) | 14.36 (40.74) | −0.96 (43.74) |
| Median | −12.9 | 5.93 | −16.2 |
| Min, Max | −61.5, 42.37 | −43.9, 79.41 | −49.5, 115.2 |

Oxidized LDL

| | | | |
|---|---|---|---|
| V2 | | | |
| N | 11 | 10 | 19 |
| Mean (SD) | 51.64 (18.34) | 48.60 (10.32) | 55.00 (17.80) |
| Median | 49.00 | 52.00 | 59.00 |
| Min, Max | 29.00, 87.00 | 28.00, 61.00 | 24.00, 86.00 |
| V6 | | | |
| N | 11 | 9 | 18 |
| Mean (SD) | 51.18 (17.62) | 54.44 (15.18) | 46.22 (15.21) |
| Median | 46.00 | 58.00 | 48.00 |
| Min, Max | 34.00, 85.00 | 25.00, 75.00 | 25.00, 77.00 |
| V2 to V6 Change | | | |
| N | 11 | 9 | 18 |
| Mean (SD) | −0.45 (5.65) | 5.22 (8.80) | −8.22 (12.33) |
| Median | 1.00 | 2.00 | −4.00 |
| Min, Max | −13.0, 6.00 | −5.00, 23.00 | −36.0, 10.00 |
| V2 to V6 % Change | | | |
| N | 11 | 9 | 18 |
| Mean (SD) | 0.41 (12.46) | 10.05 (19.84) | −12.7 (19.64) |
| Median | 2.86 | 3.92 | −10.1 |
| Min, Max | −26.0, 17.24 | −10.7, 54.76 | −43.8, 24.39 |

HDL (mg/dL)

| | | | |
|---|---|---|---|
| V2 | | | |
| N | 11 | 10 | 19 |
| Mean (SD) | 46.00 (17.37) | 48.70 (8.21) | 47.79 (14.77) |
| Median | 42.00 | 49.50 | 40.00 |
| Min, Max | 24.00, 73.00 | 34.00, 60.00 | 29.00, 77.00 |
| V6 | | | |
| N | 11 | 10 | 18 |
| Mean (SD) | 45.27 (13.68) | 48.30 (10.51) | 47.11 (14.59) |
| Median | 42.00 | 47.00 | 41.00 |
| Min, Max | 29.00, 68.00 | 38.00, 75.00 | 30.00, 75.00 |
| V2 to V6 Change | | | |
| N | 11 | 10 | 18 |
| Mean (SD) | −0.73 (5.75) | −0.40 (6.92) | −1.72 (5.19) |
| Median | −1.00 | −2.00 | −3.00 |
| Min, Max | −9.00, 7.00 | −9.00, 15.00 | −10.0, 13.00 |
| V2 to V6 % Change | | | |
| N | 11 | 10 | 18 |
| Mean (SD) | 1.78 (14.18) | −0.54 (12.87) | −3.43 (10.55) |
| Median | −2.99 | −4.11 | −5.80 |
| Min, Max | −16.3, 24.14 | −16.7, 25.00 | −22.7, 24.53 |

Table 7N provides the change in lipid levels (mg/dL) (V2 to V6) for subjects with hyperlipidemia.

TABLE 7N

| | | |
|---|---|---|
| Group X | Group Y | Group Z |
| (N = 8) | (N = 8) | (N = 15) |

Cholesterol (mg/dL)

| | | | |
|---|---|---|---|
| V2 | | | |
| N | 8 | 8 | 15 |
| Mean (SD) | 184.5 (73.91) | 182.5 (33.04) | 189.7 (58.32) |
| Median | 164.0 | 181.5 | 173.0 |
| Min, Max | 113.0, 342.0 | 141.0, 245.0 | 121.0, 316.0 |
| V6 | | | |
| N | 8 | 8 | 14 |
| Mean (SD) | 170.3 (74.95) | 194.3 (40.06) | 161.9 (46.52) |
| Median | 137.5 | 197.0 | 151.0 |
| Min, Max | 117.0, 339.0 | 124.0, 258.0 | 104.0, 284.0 |
| V2 to V6 Change | | | |
| N | 8 | 8 | 14 |
| Mean (SD) | −14.3 (17.60) | 11.75 (20.91) | −18.8 (33.97) |
| Median | −14.0 | 10.00 | −13.5 |
| Min, Max | −39.0, 8.00 | −17.0, 49.00 | −92.0, 32.00 |
| V2 to V6 % Change | | | |
| N | 8 | 8 | 14 |
| Mean (SD) | −7.65 (11.10) | 6.43 (12.94) | −9.11 (16.06) |
| Median | −6.92 | 4.64 | −8.48 |
| Min, Max | −25.0, 6.20 | −12.1, 28.16 | −38.0, 15.86 |

LDL (mg/dL)

| | | | |
|---|---|---|---|
| V2 | | | |
| N | 7 | 7 | 12 |
| Mean (SD) | 99.00 (66.54) | 100.7 (32.08) | 89.33 (40.83) |
| Median | 73.00 | 96.00 | 75.50 |
| Min, Max | 37.00, 220.0 | 63.00, 152.0 | 34.00, 166.0 |
| V6 | | | |
| N | 7 | 8 | 12 |
| Mean (SD) | 94.57 (70.92) | 107.5 (36.37) | 82.42 (37.25) |
| Median | 60.00 | 102.0 | 70.00 |
| Min, Max | 40.00, 236.0 | 65.00, 160.0 | 49.00, 176.0 |
| V2 to V6 Change | | | |
| N | 7 | 7 | 11 |
| Mean (SD) | −4.43 (12.39) | 7.29 (18.23) | −20.5 (26.25) |
| Median | −11.0 | 4.00 | −11.0 |
| Min, Max | −18.0, 16.00 | −8.00, 46.00 | −83.0, 9.00 |
| V2 to V6 % Change | | | |
| N | 7 | 7 | 11 |
| Mean (SD) | −5.19 (15.13) | 6.72 (16.95) | −17.4 (18.43) |
| Median | −10.2 | 4.94 | −12.5 |
| Min, Max | −21.6, 21.62 | −11.0, 41.82 | −50.0, 15.00 |

Triglycerides (mg/dL)

| | | | |
|---|---|---|---|
| V2 | | | |
| N | 8 | 8 | 15 |
| Mean (SD) | 235.8 (126.1) | 175.9 (121.9) | 321.9 (407.8) |
| Median | 253.5 | 148.5 | 182.0 |
| Min, Max | 80.00, 482.0 | 68.00, 444.0 | 82.00, 1677 |
| V6 | | | |
| N | 8 | 8 | 14 |
| Mean (SD) | 202.3 (120.2) | 189.3 (103.6) | 213.1 (170.3) |
| Median | 211.5 | 162.5 | 149.5 |
| Min, Max | 70.00, 420.0 | 72.00, 389.0 | 76.00, 650.0 |
| V2 to V6 Change | | | |
| N | 8 | 8 | 14 |
| Mean (SD) | −33.5 (66.92) | 13.38 (103.7) | −12.0 (124.4) |
| Median | −34.0 | 20.50 | −29.0 |
| Min, Max | −166, 56.00 | −195, 154.0 | −178, 348.0 |
| V2 to V6 % Change | | | |
| N | 8 | 8 | 14 |
| Mean (SD) | −12.9 (26.00) | 20.50 (43.68) | 0.07 (48.86) |
| Median | −14.5 | 22.11 | −21.5 |
| Min, Max | −61.5, 22.76 | −43.9, 79.41 | −49.5, 115.2 |

HDL (mg/dL)

| | | | |
|---|---|---|---|
| V2 | | | |
| N | 8 | 8 | 15 |
| Mean (SD) | 45.38 (17.08) | 49.88 (8.61) | 44.93 (13.59) |
| Median | 42.50 | 52.50 | 40.00 |
| Min, Max | 24.00, 67.00 | 34.00, 60.00 | 29.00, 77.00 |
| V6 | | | |
| N | 8 | 8 | 14 |
| Mean (SD) | 44.13 (13.24) | 48.88 (11.53) | 44.71 (13.00) |
| Median | 41.00 | 47.00 | 39.00 |
| Min, Max | 29.00, 65.00 | 38.00, 75.00 | 32.00, 72.00 |
| V2 to V6 Change | | | |
| N | 8 | 8 | 14 |
| Mean (SD) | −1.25 (6.11) | −1.00 (7.69) | −1.36 (5.64) |
| Median | −1.50 | −3.00 | −2.00 |
| Min, Max | −9.00, 7.00 | −9.00, 15.00 | −10.0, 13.00 |
| V2 to V6 % Change | | | |
| N | 8 | 8 | 14 |
| Mean (SD) | 0.85 (15.61) | −1.76 (14.26) | −2.50 (11.26) |
| Median | −3.06 | −6.78 | −4.04 |
| Min, Max | −16.3, 24.14 | −16.7, 25.00 | −22.7, 24.53 |

Table 7O provides the change in systolic and diastolic blood pressure (V2 to V6) for subjects with hypertension and all subjects.

TABLE 7O

| | | |
|---|---|---|
| Group X | Group Y | Group Z |

Systolic Blood Pressure (mmHg)

| | | | |
|---|---|---|---|
| V2 | | | |
| N | 11 | 10 | 19 |
| Mean (SD) | 137.2 (16.59) | 129.1 (12.68) | 141.9 (11.53) |
| Median | 133.0 | 131.5 | 140.0 |
| Min, Max | 110.0, 164.0 | 106.0, 144.0 | 118.0, 170.0 |
| V6 | | | |
| N | 11 | 10 | 19 |
| Mean (SD) | 126.7 (16.46) | 137.8 (26.13) | 136.2 (11.74) |
| Median | 132.0 | 129.0 | 139.0 |
| Min, Max | 99.00, 147.0 | 109.0, 191.0 | 109.0, 155.0 |
| V2 to V6 Change | | | |
| N | 11 | 10 | 19 |
| Mean (SD) | −10.5 (22.77) | 8.70 (24.84) | −5.68 (11.30) |
| Median | −11.0 | −1.00 | −6.00 |
| Min, Max | −63.0, 19.00 | −16.0, 53.00 | −22.0, 16.00 |
| V2 to V6 % Change | | | |
| N | 11 | 10 | 19 |
| Mean (SD) | −6.60 (14.70) | 7.05 (19.03) | −3.76 (7.88) |
| Median | −8.94 | −0.74 | −4.14 |
| Min, Max | −38.4, 14.84 | −11.1, 38.84 | −14.8, 11.51 |

Systolic Blood Pressure - Subjects with Hypertension (mmHg)

| | | | |
|---|---|---|---|
| V2 | | | |
| N | 10 | 9 | 19 |
| Mean (SD) | 138.6 (16.77) | 128.1 (13.03) | 141.9 (11.53) |
| Median | 137.0 | 130.0 | 140.0 |
| Min, Max | 110.0, 164.0 | 106.0, 144.0 | 118.0, 170.0 |
| V6 | | | |
| N | 10 | 9 | 19 |
| Mean (SD) | 128.2 (16.57) | 135.6 (26.67) | 136.2 (11.74) |
| Median | 132.0 | 128.0 | 139.0 |
| Min, Max | 99.00, 147.0 | 109.0, 191.0 | 109.0, 155.0 |
| V2 to V6 Change | | | |
| N | 10 | 9 | 19 |
| Mean (SD) | −10.4 (24.00) | 7.44 (26.01) | −5.68 (11.30) |
| Median | −5.00 | −3.00 | −6.00 |
| Min, Max | −63.0, 19.00 | −16.0, 53.00 | −22.0, 16.00 |
| V2 to V6 % Change | | | |
| N | 10 | 9 | 19 |
| Mean (SD) | −6.36 (15.48) | 6.22 (19.99) | −3.76 (7.88) |
| Median | −4.32 | −2.26 | −4.14 |
| Min, Max | −38.4, 14.84 | −11.1, 38.84 | −14.8, 11.51 |

Diastolic Blood Pressure (mmHg)

| | | | |
|---|---|---|---|
| V2 | | | |
| N | 11 | 10 | 19 |
| Mean (SD) | 85.09 (11.09) | 82.20 (8.22) | 85.53 (6.55) |
| Median | 83.00 | 86.00 | 85.00 |
| Min, Max | 67.00, 102.0 | 70.00, 90.00 | 72.00, 96.00 |
| V6 | | | |
| N | 11 | 10 | 19 |
| Mean (SD) | 77.27 (7.67) | 84.10 (7.89) | 80.37 (8.68) |
| Median | 80.00 | 82.00 | 80.00 |
| Min, Max | 66.00, 86.00 | 76.00, 99.00 | 64.00, 98.00 |
| V2 to V6 Change | | | |
| N | 11 | 10 | 19 |
| Mean (SD) | −7.82 (12.39) | 1.90 (7.37) | −5.16 (9.83) |
| Median | −3.00 | 4.50 | −4.00 |
| Min, Max | −32.0, 12.00 | −9.00, 10.00 | −20.0, 20.00 |
| V2 to V6 % Change | | | |
| N | 11 | 10 | 19 |
| Mean (SD) | −7.99 (13.39) | 2.76 (8.96) | −5.62 (11.81) |
| Median | −3.95 | 5.70 | −5.00 |
| Min, Max | −32.0, 16.44 | −10.1, 13.70 | −23.8, 25.64 |

Diastolic Blood Pressure - Subjects with Hypertension (mmHg)

| | | | |
|---|---|---|---|
| V2 | | | |
| N | 10 | 9 | 19 |
| Mean (SD) | 86.00 (11.24) | 83.22 (8.01) | 85.53 (6.55) |
| Median | 86.00 | 88.00 | 85.00 |
| Min, Max | 67.00, 102.0 | 70.00, 90.00 | 72.00, 96.00 |
| V6 | | | |
| N | 10 | 9 | 19 |
| Mean (SD) | 77.70 (7.94) | 84.22 (8.36) | 80.37 (8.68) |
| Median | 80.50 | 81.00 | 80.00 |
| Min, Max | 66.00, 86.00 | 76.00, 99.00 | 64.00, 98.00 |
| V2 to V6 Change | | | |
| N | 10 | 9 | 19 |
| Mean (SD) | −8.30 (12.95) | 1.00 (7.21) | −5.16 (9.83) |
| Median | −8.00 | 4.00 | −4.00 |
| Min, Max | −32.0, 12.00 | −9.00, 9.00 | −20.0, 20.00 |
| V2 to V6 % Change | | | |
| N | 10 | 9 | 19 |
| Mean (SD) | −8.39 (14.05) | 1.54 (8.58) | −5.62 (11.81) |
| Median | −8.60 | 4.55 | −5.00 |
| Min, Max | −32.0, 16.44 | −10.1, 10.00 | −23.8, 25.64 |

Table 7P provides ALT and AST levels for subjects (V2 to V6). Table 7Q provides IFN-gamma levels (pg/mL) for subjects (V2 to V6). Table 7R provides astaxanthin levels (ng/mL) for subjects at V2 and V6.

TABLE 7P

| | | |
|---|---|---|
| Group X | Group Y | Group Z |

ALT (SGPT) (U/L)

| | | | |
|---|---|---|---|
| V2 | | | |
| N | 11 | 10 | 19 |
| Mean (SD) | 39.91 (39.27) | 26.60 (10.67) | 28.47 (16.80) |
| Median | 27.00 | 25.50 | 26.00 |
| Min, Max | 19.00, 154.0 | 15.00, 41.00 | 11.00, 83.00 |
| V6 | | | |
| N | 11 | 10 | 18 |
| Mean (SD) | 26.27 (11.41) | 22.40 (3.95) | 25.50 (13.10) |
| Median | 23.00 | 23.50 | 27.00 |
| Min, Max | 12.00, 49.00 | 17.00, 28.00 | 10.00, 64.00 |
| V2 to V6 Change | | | |
| N | 11 | 10 | 18 |
| Mean (SD) | −13.6 (40.42) | −4.20 (9.27) | −2.83 (6.51) |
| Median | −1.00 | −1.00 | −1.00 |
| Min, Max | −131, 25.00 | −18.0, 6.00 | −19.0, 6.00 |
| V2 to V6 % Change | | | |
| N | 11 | 10 | 18 |
| Mean (SD) | −9.63 (51.69) | −5.19 (32.04) | −6.30 (19.02) |
| Median | −4.76 | −2.35 | −5.71 |
| Min, Max | −85.1, 125.0 | −48.5, 40.00 | −38.1, 37.50 |

AST (SGOT) (U/L)

| | | | |
|---|---|---|---|
| V2 | | | |
| N | 11 | 10 | 19 |
| Mean (SD) | 35.45 (32.29) | 20.30 (7.93) | 21.74 (6.24) |
| Median | 23.00 | 20.00 | 21.00 |
| Min, Max | 16.00, 125.0 | 9.0, 32.00 | 13.00, 37.00 |
| V6 | | | |
| N | 11 | 10 | 18 |
| Mean (SD) | 23.09 (11.20) | 18.30 (3.47) | 22.00 (7.71) |
| Median | 22.00 | 17.50 | 21.50 |
| Min, Max | 9.00, 41.00 | 14.00, 23.00 | 13.00, 37.00 |
| V2 to V6 Change | | | |
| N | 11 | 10 | 18 |
| Mean (SD) | −12.4 (34.5) | −2.00 (6.34) | 0.17 (4.71) |
| Median | −3.00 | −4.00 | −0.50 |
| Min, Max | −114, 15.00 | −9.00, 6.00 | −8.00, 10.00 |
| V2 to V6 % Change | | | |
| N | 11 | 10 | 18 |
| Mean (SD) | −12.6 (43.36) | 1.24 (37.09) | 1.08 (20.09) |
| Median | −16.7 | −20.0 | −2.50 |
| Min, Max | −91.2, 83.33 | −34.8, 66.67 | −26.7, 37.04 |

TABLE 7Q

| | Group X | Group Y | Group Z |
|---|---|---|---|
| V2 | | | |
| N | 11 | 10 | 19 |
| Mean (SD) | 11.11 (17.13) | 3.27 (2.51) | 4.45 (4.92) |
| Median | 2.63 | 2.07 | 3.60 |
| Min, Max | 1.30, 56.07 | 1.30, 8.07 | 1.30, 21.12 |
| V6 | | | |
| N | 11 | 9 | 18 |
| Mean (SD) | 4.87 (5.51) | 5.70 (3.44) | 2.72 (2.75) |
| Median | 1.30 | 6.37 | 1.30 |
| Min, Max | 1.30, 16.62 | 1.30, 10.69 | 1.30, 12.65 |
| V2 to V6 Change | | | |
| N | 11 | 9 | 18 |
| Mean (SD) | −6.24 (15.17) | 2.67 (5.32) | −1.78 (5.46) |
| Median | 0.00 | 5.07 | 0.00 |
| Min, Max | −46.0, 3.30 | −6.77, 9.39 | −19.8, 4.57 |
| V2 to V6 % Change** | | | |
| N | 11 | 9 | 18 |
| Mean (SD) | −10.6 (61.93) | 238.0 (280.0) | 4.01 (86.89) |
| Median | 0.00 | 246.1 | 0.00 |
| Min, Max | −86.8, 130.0 | −83.9, 722.3 | −93.8, 221.5 |

TABLE 7R

| | Group X | Group Y | Group Z |
|---|---|---|---|
| V2 | | | |
| N | 11 | 10 | 19 |
| Mean (SD) | 4.89 (0.00) | 9.15 (13.48) | 17.12 (49.44) |
| Median | 4.89 | 4.89 | 4.89 |
| Min, Max | 4.89, 4.89 | 4.89, 47.50 | 4.89, 221.0 |
| V6 | | | |
| N | 11 | 9 | 18 |
| Mean (SD) | 784.8 (322.2) | 9.26 (13.14) | 2586 (1529) |
| Median | 790.0 | 4.89 | 2184 |
| Min, Max | 190.9, 1282 | 4.89, 44.30 | 409.0, 7030 |

BQL (less than 9.77 ng/mL) results were treated as 4.885 for computations

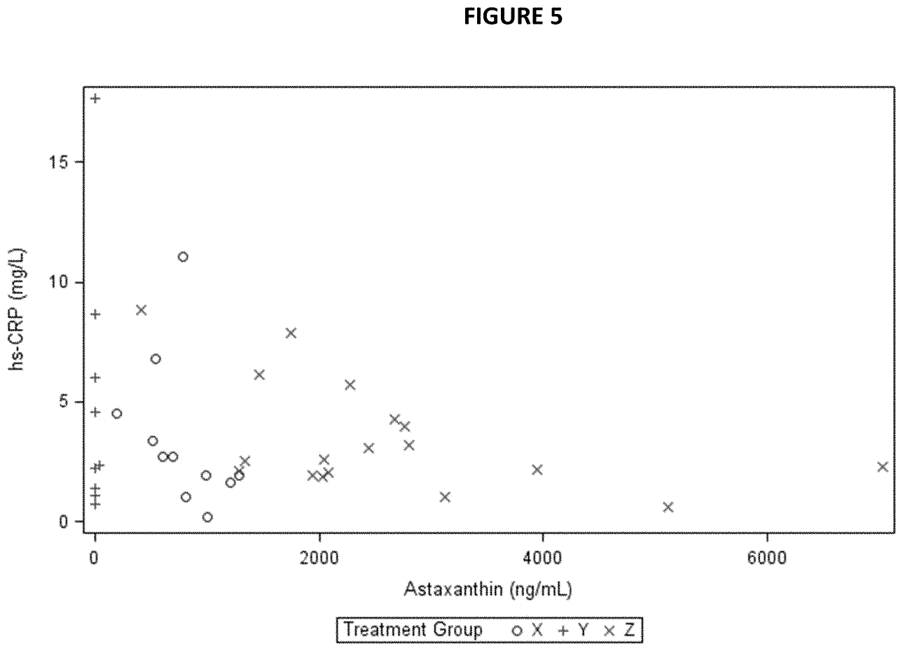

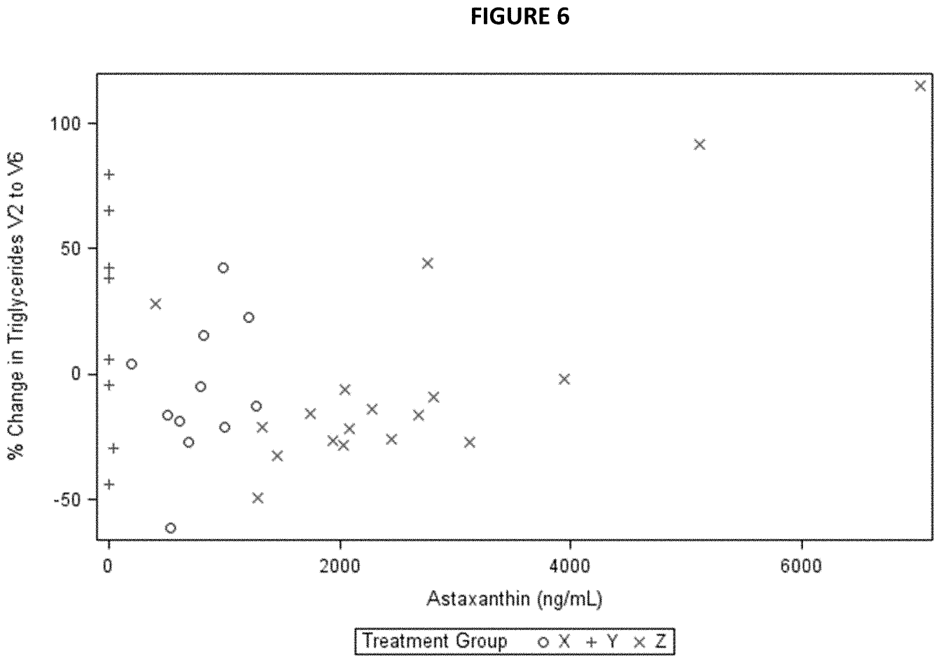

FIGS. 5 and 6 provide additional interim data from the CHASE trial. FIG. 5 is a scatterplot of hs CRP (mg/L) plotted as a function of astaxanthin concentration (ng/mL) at V6 and FIG. 6 is a scatterplot of % change in triglycerides (V2 to V6) plotted as a function of astaxanthin concentration (ng/m L).

### REFERENCES

The following references are cited throughout the application according to the number in the following listing.
1. (No author listed) “Screening for asymptomatic coronary artery disease. U.S. Preventive Services Task Force.” Am Fam Physician. 1989 December; 40(6):99-104.
2. Ridker, P M, “The JUPITER Trial, Results, Controversies, and Implications for Prevention,” Circ Cardiovas Qual Outcomes. 2009; 2:279-285.
3. Ridker, P M, et al, “Antiinflammatory Therapy with Canakinumab for Atherosclerotic Disease,” N engl J Med. 2017 Sep. 21; 377(12):1119-1131.
4. Baylis R A et al, “The CANTOS Trial: One Important Step for Clinical Cardiology but a Giant Leap for Vascular Biology,” Arterioscler Thromb Vasc Biol. 2017 November; 37(11):e174-e177.
5. Everett B M et al., “Rationale and Design for the Cardiovascular Inflammation Reduction Trial (CIRT): A test of the Inflammatory Hypothesis of Atherothrombosis. Am Heart J. 2013 August; 166(2): 199-207.
6. Kidd P. “Astaxanthin, cell membrane nutrient with diverse clinical benefits and anti-aging potential.” Altern Med Rev. 2011 December; 16(4): 355-64.
7. Fassett R B, Coombes J S. “Astaxanthin in Cardiovascular Health and Disease.” Molecules. 2012; 17: 2030-48.
8. Shimidzu N. “Carotenoids as singlet oxygen quenchers in marine organisms.” Fish Sci. 1996; 62: 134-7.
9. Krinsky N I. “Antioxidant functions of carotenoids.” Free Radic Biol Med. 1989; 7: 617-35.
10. Miki W. “Biological functions and activities of animal carotenoids.” Pure Appl. Chem. 1991; 63: 141-6.
11. Tanaka T, et al. “Cancer Chemoprevention by Carotenoids.” Molecules. 2012; 17: 3202-42.
12. Pashkow F J, et al. “Astaxanthin: a novel potential treatment for oxidative stress and inflammation in cardiovascular disease.” Am J Cardiol. 2008 May 22; 101(10A): 58D-68D.
13. Edwards J A, et al. “Review of genotoxicity and rat carcinogenicity in investigations with astaxanthin.” Regul Toxicol Pharmacol. 2016: 75: 5-19.
14. Fassett R G and Coombes J S, “Astaxanthin: A Potential Therapeutic Agent in Cardiovascular Disease.” Mar. Drugs 2011, 9, 447-465.
15. Ohno M, Darwish W S, et al “Astaxanthin can alter CYP1A-dependent activities via two different mechanisms: induction of protein expression and inhibition of NADPH P450 reductase dependent electron transfer.” Food Chem Toxicol. 2011 June; 49(6):1285-91.
16. Kistler A, et al “Metabolism and CYP-inducer properties of astaxanthin in man and primary human hepatocytes.” Arch Toxicol. 2002 January; 75(11-12):665-75.
17. Curek G D, et al “Effect of astaxanthin on hepatocellular injury following ischemia/reperfusion.” Toxicology. 2010 Jan. 12:267(1-3):147-53.
18. Zhang J, Zhang S, et al “Astaxanthin pretreatment attenuates acetaminophen-induced liver injury in mice.” Int Immunopharmacol. 2017 April; 45:26-33.
19. Otsuka T, Shimazawa M, et al “Astaxanthin Protects Against Retinal Damage: Evidence from In Vivo an dln Vitro Retinal ischemia and Reperfusion Models.” Curr Eye Res. 2016 November; 41(11):1465-1472.
20. Zheng D, Li Y, et al. “The protective effect of astaxanthin on fetal alcohol spectrum disorder in mice.” Neuropharmacology. 2014 September; 84:13-8.
21. Takizawa Y, Kitazato T, et al. “Effects of antioxidants on drug absorption in in vivo intestinal ischemia/reperfusion.” Eur J Drug Metab Pharmacokinet. 2011 January; 35(3-4):89-95.
22. Qiu X, Fu K, et al. “Protective effects of astaxanthin against ischemia/reperfusion induced renal injury in mice.” J Transl Med. 2015 Jan. 27; 13:28.
23. Polotow T G, Vardaris C V, et al “Astaxanthin supplementation delays physical exhaustion and prevents redox imbalances in plasma and soleus muscles of Wistar rats.” Nutrients. 2014 Dec. 12; 6(12):5819-38.
24. Xu L, Zhu J, et al. “Astaxanthin improves cognitive deficits from oxidative stress, nitric oxide synthase and inflammation through upregulation of PI3K/Akt in diabetes rat.” Int J Clin Exp Pathol. 2015 Jun. 1; 8(6):6083-94.
25. Abdelzaher L A, Imaizume T, et al “Astaxanthin alleviates oxidative stress insults-related derangements in human vascular endothelial cells exposed to glucose fluctuations.” Life Sci 2016 Apr. 1; 150:24-31.
26. Martins, Rute, Lithgow, Gordon J, Link, Wolfang, “Long live FOXO: unraveling the role of FOXO proteins in aging and longevity.” Aging Cell (2016) 15, pp 196-207.
27. Tia N, Singh A K, et al. “The Role of Forkhead Box 0 (FOXO) transcription factor in aging and disease.” Gene. 2018 Mar. 30; 648:97-105.
28. Coral-Hinostroza G N, et al. “Plasma appearance of unesterified Astaxanthin geometrical E/Z and optical R/S isomers in men given single doses of a mixture of optical 3 and 3′R/S isomers of Astaxanthin fatty acyl diesters.” Comp Biochem Physkol C Toxicol Pharmacol. 2004 October; 139: 1-3.
29. Okada Y, et al. “Oral bioavailability of Astaxanthin in Haematococcus Algal Extract: The Effects of Timing of Diet and Smoking Habits.” Biosci Biotechnol Biochem. 2009; 73(9), 1928-32.
30. Odeberg J H, et al. “Oral bioavailability of the antioxidant Astaxanthin in humans is enhanced by incorporation of lipid based formulations.” Eur J Pharm Sci. 2003; 19(4): 299-304.
31. Choi H D, et al. “Effects of Astaxanthin on Oxidative Stress in Overweight and Obese Adults.” Phytother Res. 2011 December; 25(12): 1813-8.
32. Miyazawa T, et al. “Plasma Carotenoid Concentrations before and after Supplementation with Astaxanthin in Middle Aged and Senior Subjects.” Biosci Bioechnol Biochem. 2011; 75(9): 1856-8.
33. Spiller G A, Dewell A. “Safety of an astaxanthin-rich Haematococcus pluvialis algal extract: A randomized clinical trial.” J Med Food. 2003 Spring; 6(1):51-6.
34. Kishimoto Y, Yoshida H, Kondo K, “Potential Anti-Atherosclerotic Properties of Astaxanthin,” March Drugs. 2016 Feb. 5; 142(2).
35. Uchiyama, Akiyoshi, and Okada, Yumika, “Clinical Efficacy of Astaxanthin-containing Haematococcus Pluvialis Extract for the Volunteers at Risk of Metabolic Syndrome.” J. Clini. Biochem. Nutr., 43 Suppl. 1, 38-43, July 2008.
36. U S Department of Health and Human Services, Food and Drug Administration, Center for Drug Evaluation and Research “Guidance for Industry: Estimating the Maximum Safe Starting Dose in Initial Clinical Trials for Therapeutics in Adult Healthy Volunteers.” July 2005.
37. Food and Drug Administration. “Guidance for Clinical Investigators, Sponsors, and IRBs Investigational New Drug Applications (INDs)—Determining Whether Human Research Studies Can Be Conducted Without an IND.” September 2013.
38. Iwamoto, T, hosodo K, et al. “Inhibition of low-density lipoprotein oxidation by astaxanthin.” J Atheroscler Thromb. 2000; 7(4):216-22
39. Osterlie, M, Bjerkeng B, Liaaen-Jensen S. “Plasma appearance and distribution of astaxanthin E/Z and R/S isomers in plasma lipoproteins of men after single dose administration of astaxanthin.” J Nutr Biochem. 2000 October; 11(10):482-90.
40. Karppi, J, Rissanen T H, et al. “Effects of astaxanthin supplementation on lipid peroxidation.” Int J Vitam Nutr Res. 2007 January; 77(1):3-11.
41. Parisi V, Tedeschi M, et al. “Carotenoids and antioxidants in age-related maculopathy Italian study: multifocal electroretinogram modifications after 1 year.” Ophthalmology. 2008 February; 115(2):324-333.
42. Miyawaki H, Takahashi J, et al. “Effects of astaxanthin on human blood rheology.” J Clin Biochem Nutr. 2008 September; 43(2):69-74.
43. Rufer C E, Moeseneder J, et al. “Bioavailability of astaxanthin stereoisomers from wild (Oncorhynchus spp.) and aquacultured (Salmo salar) salmon in healthy men: a randomized, double-blind study. Br J Nutr. 2008 May; 99(5):1048-54.
44. Park, J S, Chyun J H, et al, “Astaxanthin decreased oxidative stress and inflammation and enhanced immune response in humans.” Nutrition & Metabolism 2010, 7:18.
45. Choi H D, Youn Y K, Shin W G, “Positive Effects of Astaxanthin on Lipid Profiles and Oxidative Stress in Overweight Subjects.” Plant Foods Human Nutr. 2011 November; 66(4):363-9.
46. Satoh Akira, Tsuji Shinji, et al. “Preliminary Clinical Evaluation of Toxicity and Efficacy of A New Astaxanthin-rich Haematococcus pluvialis Extract.” J. Clin. Bkochem. Nutr., 44, 280-284, May 2009
47. Klinkenberg, L J J, Res, Peter T, et al, “Effect of Antioxidant Supplementatkon on Exercise-Induced Cardiac Troponin Release in Cyclists: A Randomized Trial.” PLOS ONE, November 2013, Vol 8, Issue 11, e79280.
48. Earnest, C P, Lupo M, et al. “Effect of astaxanthin on cycling time trial performance.” Int J Sports Med. 2011 November; 32(11):882-8
49. Comhaire, F J, El Garem Y, et al, “Combined conventional/antioxidant “Astaxanthin” treatment for male infertility: a double-blind, randomized trial. Asian J Androl 2005: 7(3): 257-262.
50. Coombs J S, Sharman J E, Fassett R G, “Astaxanthin has no effect on arterial stiffness, oxidative stress, or inflammation in renal transplan recipients: a randomized controlled study (the XANTHIN trial). Am J Clin Nutr. 2016 January; 103(1)283-9.
51. Piermarocchi S, Saviano S, et al. “Carotenoids in Age-related Maculopathy Italian Study (CARMIS): two-year results of a randomized study.” Eur J Ophthalmol. 2012 March-April; 22(2):216-25.
52. Saito M, Yoshida K, et al. “Astaxanthin increases choroidal blood flow velocity.” Graefes Arch Clin Exp Ophthalmol. 2012 February; 250(2):239-45.
53. Kim J H, Chang M J, et al. “Protective effects of Haematococcus astaxanthin on oxidative stress in healthy smokers.” J Med Food. 2011 November; 14(11):1469-75.
54. Kupcinskas L, Lafolie P, et al. “Efficacy of the natural antioxidant astaxanthin in the treatment of functional dyspepsia in patients with or without Helicobactor pylori infection: A prospective, randomized, double blind, and placebo-controlled study.” Phytomedicine. 2008 June; 15(6-7):391-9.
55. Tominaga, Kumi, Hongo Nobuko, et al. “Cosmetic benefits of astaxanthin on human subjects.” Acta ABP 2012, 59(1) 43-47.
56. Tominaga K, Hongo N, et al. “Protective effects of astaxanthin on skin deterioration.” J Clin Biochem Nutr. 2017 July; 61(1):33-39.
57. Nakagawa, K, Kiko T, et al. “Antioxidant effect of astaxanthin on phospholipid peroxidation in human erythrocytes.” Br J of Nutr (2011), 105, 1563-1571.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of preventing or treating cardiovascular disease in a subject or supporting cardiovascular health or improving inflammatory health in a subject at risk for or suffering from cardiovascular disease, by reducing C-reactive protein (CRP) levels comprising administering a therapeutically effective amount of astaxanthin to the subject, wherein the subject also receives standard of care for cardiovascular disease or risk management.

2. (canceled)

3. (canceled)

4. The method of claim 1, wherein the amount of astaxanthin administered is 24 mg/day, optionally as 12 mg twice a day.

5. The method of claim 1, wherein the amount of astaxanthin administered is 96 mg/day, optionally as 48 mg twice a day.

6. The method of claim 1, wherein the amount of astaxanthin administered results in a level of astaxanthin in the bloodstream in the range of 0.1 to 1 μg/mL in plasma or serum or in the range of 1 to 10 μg/mL in plasma or serum.

7. (canceled)

8. A method of reducing C-reactive protein (CRP) levels in a subject at risk for or suffering from cardiovascular disease and optionally receives standard of care for cardiovascular disease or risk management, said method comprising administering astaxanthin (A) at an amount of 24 mg/day, optionally as 12 mg twice a day, (B) at an amount of 96 mg/day, optionally as 48 mg twice a day, (C) at an amount that results in a therapeutic level of astaxanthin in the bloodstream in the range of 0.1 to 1 μg/mL in plasma or serum, or (D) at an amount that results in a therapeutic level of astaxanthin in the bloodstream in the range of 1 to 10 μg/mL in plasma or serum.

9. (canceled)

10. (canceled)

11. (canceled)

12. The method of claim 1, wherein the subject, prior to the administration of astaxanthin, exhibits a C-reactive protein (CRP) level of at least 2 mg/L or following the administration of astaxanthin, exhibits a C-reactive protein (CRP) level below 2 mg/L.

13. (canceled)

14. A method of (a) reducing levels of one or more of the following: TNF-α, IL-1β, IL-6, INF-γ, oxidized LDL, total cholesterol, LDL cholesterol, VLDL cholesterol, triglycerides, HbA1c, ALT, AST, body weight, or blood pressure; or (b) increasing levels of FOXO3 activation and/or HDL cholesterol; in a subject comprising administering astaxanthin, wherein the subject also receives standard of care for cardiovascular disease or risk management, optionally, wherein astaxanthin is administered at (A) an amount of 24 mg/day or 12 mg twice a day, wherein the subject is at risk for or suffers from cardiovascular disease, (B) at an amount of 96 mg/day or 48 mg twice a day, wherein the subject is at risk for or suffers from cardiovascular disease, (C) at an amount that results in a therapeutic level of astaxanthin in the bloodstream in the range of 0.1 to 1 μg/mL in plasma or serum, wherein the subject is at risk for or suffers from cardiovascular disease or (D) at an amount that results in a therapeutic level of astaxanthin in the bloodstream in the range of 1 to 10 μg/mL in plasma or serum, wherein the subject is at risk for or suffers from cardiovascular disease.

15.-18. (canceled)

19. The method of claim 1, wherein the subject has (or had) one of more of the following: myocardial infarction, coronary artery bypass graft (CABG), angioplasty, an intra-coronary artery stent, angina, stroke, transient ischemic attack, atherosclerotic carotid artery disease.

20. The method of claim 1, wherein the subject suffers from Diabetes Mellitus Type II, hypertension, hyperlipidemia, or obesity, or is a smoker.

21. The method of claim 1, wherein the standard of care for cardiovascular disease or risk management comprises administration of a cardiac therapeutic agent.

22. The method of claim 21, wherein the cardiac therapeutic agent is one or more of a statin, a hypertension medication, niacin, fibrate, CETP inhibitor, an anti-diabetic agent, and a vasoactive agent or comprises a diuretic, a beta blocker, an ACE inhibitor, an angiotensin receptor blocker, a calcium channel blocker, an alpha blocker, an alpha-2 receptor agonist, a central agonist, a renin inhibitor, an arterial vasodilator, or a combination thereof.

23. (canceled)

24. The method of claim 1, wherein the standard of care for cardiovascular disease or risk management comprises administration of an anti-diabetic or anti-obesity agent.

25. The method of claim 1, wherein the effect is demonstrated within 1 to 2 weeks, within 4 weeks, within 8 weeks, within 12 weeks, within 24 weeks, or within 48 weeks.

26.-30. (canceled)

31. The method of claim 1, wherein the astaxanthin is administered within about 30 minutes after food (e.g., a meal).

32. The method of claim 1, wherein the astaxanthin is 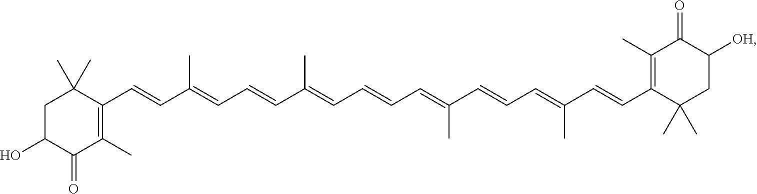 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]
or a pharmaceutically acceptable salt thereof.

33. The method of claim 32, wherein the astaxanthin is a mixture of (S,S′), (R,R′), and meso isomer forms of astaxanthin.

34. The method of claim 32, wherein the astaxanthin is synthetically produced.

35. The method of claim 1, wherein the subject's AST and/or ALT levels are not increased upon administration of astaxanthin.

36. The method of claim 1, wherein the subject exhibits a decreased non-HDL cholesterol and/or wherein the subject is not taking a statin, or is not taking a cardiac therapeutic agent, or is not taking a hypertension medication.

37. (canceled)
